(19) United States
(12) Patent Application Publication
(10) Pub. No.: US 2010/0132070 A1
(43) Pub. Date: May 27, 2010

(54) Plant Defense Genes and Proteins and Methods of Use

(75) Inventors: James English, San Leandro, CA (US); Carl R. Simmons, Des Moines, IA (US); Nasser Yalpani, Johnston, IA (US)

Correspondence Address: PIONEER HI-BRED INTERNATIONAL, INC., 7250 N.W. 62ND AVENUE, P.O. BOX 552, JOHNSTON, IA, 50131-0552, US

(73) Assignee: Pioneer Hi-Bred International, Inc.

(21) Appl. No.: 12/618,829

(22) Filed: Nov. 16, 2009

Related U.S. Application Data
(60) Provisional application No. 61/116,650, filed on Nov. 21, 2008.

(51) Int. Cl. A01H 5/00; C07H 21/04; C12N 5/10; C12N 15/82

(52) U.S. Cl. 800/279; 536/23.1; 435/419; 800/298; 800/301

(57) ABSTRACT

Methods and compositions for modulating development and defense responses are provided. Nucleotide sequences encoding plant defense proteins are provided. The sequences can be used in expression cassettes for modulating development, developmental pathways, and defense responses. Transformed plants, plant cells, tissues, and seed are also provided.

### CROSS-REFERENCE TO RELATED APPLICATIONS

This application claims the benefit of U.S. Provisional Application Ser. No. 61/116,650, filed Nov. 21, 2008, the content of which is herein incorporated by reference in its entirety.

### FIELD OF INVENTION

The invention relates to the field of the genetic manipulation of plants, particularly the modulation of gene activity and development in plants and increased disease resistance.

### BACKGROUND OF THE INVENTION

Disease in plants is caused by biotic and abiotic causes. Biotic causes include fungi, viruses, bacteria, and nematodes. An example of the importance of plant disease is illustrated by phytopathogenic fungi, which cause significant annual crop yield losses as well as devastating epidemics. Plant disease outbreaks have resulted in catastrophic crop failures that have triggered famines and caused major social change. All of the approximately 300,000 species of flowering plants are attacked by pathogenic fungi; however, a single plant species can be host to only a few fungal species, and similarly, most fungi usually have a limited host range. Generally, the best strategy for plant disease control is to use resistant cultivars selected or developed by plant breeders for this purpose. However, the potential for serious crop disease epidemics persists today, as evidenced by outbreaks of the Victoria blight of oats and southern corn leaf blight. Molecular methods of crop protection have the potential to implement novel mechanisms for disease resistance and can also be implemented more quickly than traditional breeding methods. Accordingly, molecular methods are needed to supplement traditional breeding methods to protect plants from pathogen attack.

Recently, agricultural scientists have developed crop plants with enhanced pathogen resistance by genetically engineering plants to express antipathogenic proteins. For example, potatoes and tobacco plants genetically engineered to produce an antifungal endochitinase protein were shown to exhibit increased resistance to foliar and soil-borne fungal pathogens. See Lorito et al. (1998) Proc. Natl. Acad. Sci. 95:7860-7865. Moreover, transgenic barley that is resistant to the stem rust fungus has also been developed. See Horvath et al. (2003) Proc. Natl. Acad. Sci. 100:364-369. A continuing effort to identify antipathogenic agents and to genetically engineer disease-resistant plants is underway.

Thus, in light of the significant impact of plant pathogens, particularly fungal pathogens, on the yield and quality of crops, new compositions and methods for protecting plants from pathogens are needed. Methods and compositions for controlling multiple fungal pathogens are of particular interest.

### SUMMARY OF THE INVENTION

Compositions and methods relating to pest resistance are provided. Particularly, nucleic acid molecules and amino acid sequences for defense of plants are provided. The nucleotide sequences of the invention encode proteins that are variously annotated or described, and which include, but are not limited to: defensins, defensin-like proteins, antimicrobial peptides, anti-pathogenic peptides, thionins, antifungal peptides, protease inhibitors, proteinase inhibitors, subtilisin or chymotrypsin inhibitors, amylase inhibitors, or scorpion toxin-like proteins.

The genes of the present invention may find use in enhancing the plant pathogen defense system, and are referred to herein as plant defense genes and proteins. The compositions and methods of the invention can be used for enhancing resistance to plant pathogens including fungal pathogens, microorganisms, nematodes, insects, and the like. The method involves stably transforming a plant with a nucleotide sequence capable of modulating the plant pathogen defense system operably linked with a promoter capable of driving expression of a gene in a plant cell. These plant defense genes additionally find use in manipulating these processes in transformed plants and plant cells.

Transformed plants, plant cells, and seeds, as well as methods for making such plants, plant cells, and seeds are additionally provided. It is recognized that a variety of promoters will be useful in the invention, the choice of which will depend in part upon the desired level of expression of the disclosed genes. It is recognized that the levels of expression can be controlled to modulate the levels of expression in the plant cell.

### DETAILED DESCRIPTION OF THE INVENTION

### Overview

The present invention provides, inter alia, compositions and methods for modulating the total level of polypeptides of the present invention and/or altering their ratios in a plant. “Modulation” is used to mean an increase or decrease in a particular character, quality, substance, or response.

The compositions comprise nucleotide and amino acid sequences from numerous plant species. Particularly, the nucleotide and amino acid sequences for numerous plant defense proteins are provided. “Plant defense genes” are genes that include defensins, defensin-like proteins, antimicrobial peptides, anti-pathogenic peptides, thionins, antifungal peptides, protease inhibitors, proteinase inhibitors, subtilisin or chymotrypsin inhibitors, amylase inhibitors, or scorpion toxin-like proteins They are called Plant defense genes because they play a role in defense, more specifically plant defense against pathogens. While not bound by any mechanism of action, expression of the sequences and related genes around disease induced lesions may control symptom development, as in a hypersensitive response (HR), by controlling the protease mediated cell death mechanism. The compositions may also function directly as antipathogenic proteins by inhibiting proteases produced by pathogens or by binding cell wall components of pathogens. Thirdly, they may also act as amphipathic proteins that perturb membrane function, leading to cellular toxicity of the pathogens. The plant defense genes generally demonstrate antimicrobial activity. “Antimicrobial” or “antimicrobial activity” means antibacterial, antiviral, nematocidal, insecticidal, or and antifungal activity. Accordingly, the polypeptides of the invention may enhance resistance to insects and nematodes. Any one plant defense gene exhibits a spectrum of antimicrobial activity that may involve one or more antibacterial, antifungal, antiviral, insecticidal, nematocidal, or antipathogenic activities. They may also be useful in regulating seed storage protein turnover and metabolism.

The plant defense genes of the invention encode plant defense peptides that inhibit the growth of a broad range of pathogens, including but not limited to fungi, nematocides, bacteria, and insects at micromolar concentrations, and can also enhance the plant's natural pathogen defense system. Thus, “plant defense peptide activity” means that the peptides inhibit pathogen growth or damage caused by a variety of pathogens, including but not limited to, fungi, insects, nematodes and bacteria. Plant defense genes inhibit pathogen damage through a variety of mechanisms including, but not limited to, alteration of membrane ion permeability and induction of hyphal branching in fungal targets (Garcia-Olmeda et al. (1998) Peptide Science 47:479-491, herein incorporated by reference).

The compositions of the invention can be used in a variety of methods whereby the protein products can be expressed in crop plants to function as plant defense proteins. Expression will result in alterations or modulation of the level, tissue, or timing of expression to achieve enhanced disease, insect, nematode, viral, fungal, or stress resistance. The compositions of the invention may be expressed in the native species including, but not limited to Nicotiana benthamiana, Vernonia mespilifolia, Triticum aestivum, Zea mays, Tulipa gesneriana, Beta vulgaris, Amaranthus retroflexus, Hedera helix, or alternatively, can be heterologously expressed in any plant of interest. In this manner, the coding sequence for the plant defense protein can be used in combination with a promoter capable of driving expression in a plant cell, that is introduced into a crop plant to enhance resistance. The phrase “enhancing resistance” means increasing the tolerance of the plant to pathogens. In other words, the plant defense gene may slow or prevent pathogen infection and/or spread.

In one embodiment, a high-level expressing constitutive promoter may be utilized and would result in high levels of expression of the plant defense protein. In other embodiments, the coding sequence may be operably linked to a tissue-preferred promoter to direct the expression to a plant tissue known to be susceptible to a pathogen. Likewise, manipulation of the timing of expression may be utilized. For example, by judicious choice of promoter, expression can be enhanced early in plant growth to prime the plant to be responsive to pathogen attack. Likewise, pathogen inducible promoters can be used wherein expression of the plant defense gene is turned on in the presence of the pathogen.

If desired, a transit peptide can be utilized to direct cellular localization of the protein product. In this manner, the native transit peptide or a heterologous transit peptide can be used. However, it is recognized that both extracellular expression and intracellular expression are encompassed by the methods of the invention.

Sequences of the invention, as discussed in more detail below, encompass coding sequences, antisense sequences, and fragments and variants thereof. Expression of the sequences of the invention can be used to modulate or regulate the expression of corresponding plant defense proteins.

### Compositions

Compositions of the invention include nucleotide sequences that have been identified as plant defense genes. Plant defense genes are involved in defense response and development. In particular, the present invention provides for isolated nucleic acid molecules comprising nucleotide sequences encoding the amino acid sequences shown in SEQ ID NOs: 4, 5, 9, 10, 14, 15, 19, 20, 24, 25, 29, 30, 34, 35, 39, 40, 44, 45, 49, 50, 53, 54, 58, 59, 63, 64, 68, 69, 73, 74, 78, 79, 83, 84, 88, 89, 93, 94, 98, 99, 103, 104, 108, 109, 113, 114, 118, 119, 120, 121, 122, 123 and 124. In particular the invention provides the mature polypeptides having the amino acid sequences set forth in SEQ ID NOs: 5, 10, 15, 20, 25, 30, 35, 40, 45, 50, 54, 59, 64, 69, 74, 79, 34, 89, 94, 99, 104, 109, 114, and 119. Further provided are polypeptides having an amino acid sequence encoded by a nucleic acid molecule described herein, for example those set forth in SEQ ID NOs: 1, 2, 3, 6, 7, 8, 11, 12, 13, 16, 17, 18, 21, 22, 23, 26, 27, 28, 31, 32, 33, 36, 37, 38, 41, 42, 43, 46, 47, 48, 51, 52, 55, 56, 57, 60, 61, 62, 65, 66, 67, 70, 71, 72, 75, 76, 77, 80, 81, 82, 85, 86, 87, 90, 91, 92, 95, 96, 97, 100, 101, 102, 105, 106, 107, 110, 111, 112, 115, 116, and 117.

The invention encompasses isolated or substantially purified nucleic acid or protein compositions. An “isolated” or “purified” nucleic acid molecule or protein, or biologically active portion thereof, is substantially or essentially free from components that normally accompany or interact with the nucleic acid molecule or protein as found in its naturally occurring environment. Thus, an isolated or purified nucleic acid molecule or protein is substantially free of other cellular material or culture medium when produced by recombinant techniques, or substantially free of chemical precursors or other chemicals when chemically synthesized. Preferably, an “isolated” nucleic acid is free of sequences (preferably protein encoding sequences) that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, or 1% (by dry weight) of contaminating protein. When the protein of the invention or biologically active portion thereof is recombinantly produced, preferably culture medium represents less than about 30%, 20%, 10%, 5%, or 1% (by dry weight) of chemical precursors or non-protein-of-interest chemicals.

Fragments and variants of the disclosed nucleotide sequences and proteins encoded thereby are also encompassed by the present invention. A “fragment” is a portion of the nucleotide sequence or a portion of the amino acid sequence and hence protein encoded thereby. Fragments of a nucleotide sequence may encode protein fragments that retain the biological activity of the native protein and hence have plant defense activity and thereby affect development, developmental pathways, and defense responses. Alternatively, fragments of a nucleotide sequence that are useful as hybridization probes generally do not encode fragment proteins retaining biological activity. Thus, fragments of a nucleotide sequence may range from at least about 20 nucleotides, about 50 nucleotides, about 100 nucleotides, and up to the full-length nucleotide sequence encoding the proteins of the invention.

A fragment of a plant defense nucleotide sequence that encodes a biologically active portion of a plant defense protein of the invention will encode at least 15, 25, 30, 40, 50, 60, 70, 80, 90, 100, 110, 120, 130, 140, 150, 160, 170, or 175 contiguous amino acids, or up to the total number of amino acids present in a full-length protein of the invention. Fragments of a plant defense gene nucleotide sequence that are useful as hybridization probes for PCR primers generally need not encode a biologically active portion of a plant defense protein.

Thus, a fragment of a plant defense nucleotide sequence may encode a biologically active portion of a plant defense protein, or it may be a fragment that can be used as a hybridization probe or PCR primer using methods disclosed below. A biologically active portion of a plant defense protein can be prepared by isolating a portion of one of the plant defense nucleotide sequences of the invention, expressing the encoded portion of the plant defense protein (e.g., by recombinant expression in vitro), and assessing the activity of the encoded portion of the plant defense protein. Nucleic acid molecules that are fragments of a plant defense nucleotide sequence comprise at least 16, 20, 50, 75, 100, 150, 200, 250, 300, 350, 400, 450, 500, 550, 600, 650, 700, 750, 800, 850, 900 or 950 nucleotides, or up to the number of nucleotides present in a full-length plant defense nucleotide sequences disclosed herein.

A “variant” is a substantially similar sequence. For nucleotide sequences, conservative variants include those sequences that, because of the degeneracy of the genetic code, encode the amino acid sequence of one of the plant defense polypeptides of the invention. Naturally occurring allelic variants such as these can be identified with the use of well-known molecular biology techniques, as, for example, with polymerase chain reaction (PCR) and hybridization techniques as outlined below. Variant nucleotide sequences also include synthetically derived nucleotide sequences, such as those generated, for example, by using site-directed mutagenesis but which still encode a plant defense protein of the invention. Generally, variants of a particular nucleotide sequence of the invention will have at least about 50%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to that particular nucleotide sequence as determined by sequence alignment programs described elsewhere herein using default parameters.

A “variant protein” is a protein derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein may be present in a variant protein. Variant proteins encompassed by the present invention are biologically active, that is they continue to possess the desired biological activity of the native protein, that is, plant defense activity as described herein. Such variants may result from, for example, genetic polymorphism or from human manipulation. Biologically active variants of a native plant defense protein of the invention will have at least about 40%, 50%, 60%, 65%, 70%, generally at least about 75%, 80%, 85%, preferably at least about 90%, 91%, 92%, 93%, 94%, 95%, 96%, 97%, and more preferably at least about 98%, 99% or more sequence identity to the amino acid sequence for the native protein as determined by sequence alignment programs described elsewhere herein using default parameters. A biologically active variant of a protein of the invention may differ from that protein by as few as 1-15 amino acid residues, as few as 1-10, such as 6-10, as few as 5, as few as 4, 3, 2, or even 1 amino acid residue.

Biological activity of the plant defense polypeptides (i.e., influencing the plant defense response and various developmental pathways, including, for example, influencing cell division) can be assayed by any method known in the art. Biological activity of the polypeptides of the present invention can be assayed by any method known in the art (see for example, U.S. Pat. No. 5,614,395; Thomma et al. (1998) Plant Biology 95:15107-15111; Liu et al. (1994) Plant Biology 91:1888-1892; Hu et al. (1997) Plant Mol. Biol. 34:949-959; Cammue et al. (1992) J. Biol. Chem. 267:2228-2233; and Thevissen et al. (1996) J. Biol. Chem. 271:15018-15025, all of which are herein incorporated by reference). Furthermore, assays to detect plant defense gene activity include, for example, assessing antifungal and/or antimicrobial activity (Terras et al. (1992) J. Biol. Chem. 267:14301-15309; Terras et al. (1993) Plant Physiol (Bethesda) 103:1311-1319; Terras et al. (1995) Plant Cell 7:573-588, Moreno et al. (1994) Eur. J. Biochem. 223:135-139; and Osborn et al. (1995) FEBS Lett. 368:257-262, all of which are herein incorporated by reference).

The polypeptides of the invention may be altered in various ways including amino acid substitutions, deletions, truncations, and insertions. Novel proteins having properties of interest may be created by combining elements and fragments of proteins of the present invention as well as other proteins. Methods for such manipulations are generally known in the art. For example, amino acid sequence variants of the plant defense proteins can be prepared by mutations in the DNA. Methods for mutagenesis and nucleotide sequence alterations are well known in the art. See, for example, Kunkel (1985) Proc. Natl. Acad. Sci. USA 82:488-492; Kunkel et al. (1987) Methods in Enzymol. 154:367-382; U.S. Pat. No. 4,873,192; Walker and Gaastra, eds. (1983) Techniques in Molecular Biology (Macmillan Publishing Company, New York) and the references cited therein. Guidance as to appropriate amino acid substitutions that do not affect biological activity of the protein of interest may be found in the model of Dayhoff et al. (1978) Atlas of Protein Sequence and Structure (Natl. Biomed. Res. Found., Washington, D.C.), herein incorporated by reference. Conservative substitutions, such as exchanging one amino acid with another having similar properties, may be preferred.

Thus, the genes and nucleotide sequences of the invention include both naturally occurring sequences as well as mutant forms. Likewise, the proteins of the invention encompass naturally occurring proteins as well as variations and modified forms thereof, such as shuffled variant peptides. Such variants will continue to possess the desired developmental activity, or plant defense response activity. Obviously, the mutations that will be made in the DNA encoding the variant must not place the sequence out of reading frame and preferably will not create complementary regions that could produce secondary mRNA structure. See, for example, EP Patent Application Publication No. 75,444.

The deletions, insertions, and substitutions of the protein sequences encompassed herein are not expected to produce radical changes in the characteristics of the protein. However, when it is difficult to predict the exact effect of the substitution, deletion, or insertion in advance of doing so, one skilled in the art will appreciate that the effect will be evaluated by routine screening assays. That is, the activity can be evaluated by activity assays. See, for example, Lancaster et al. (1994) J. Biol. Chem. 14:1137-1142 and Terras et al. (1995) Plant Cell 7:537-588, herein incorporated by reference. Additionally, differences in the expression of specific genes between uninfected and infected plants can be determined using gene expression profiling. RNA may be analyzed, for example, using the gene expression profiling process (GeneCalling®) as described in U.S. Pat. No. 5,871,697.

Variant nucleotide sequences and proteins also encompass sequences and proteins derived from a mutagenic and recombinogenic procedure such as DNA shuffling. SEQ ID NOs: 120, 121, 122, 123 and 124 are shuffled sequences. Any other sequences of the invention could also be subjected to shuffling procedures to create variant sequences. In shuffling, one or more different plant defense protein coding sequences is manipulated to create a new plant defense protein possessing the desired properties. In this manner, libraries of recombinant polynucleotides are generated from a population of related sequence polynucleotides comprising sequence regions that have substantial sequence identity and can be homologously recombined in vitro or in vivo. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458.

The nucleotide sequences of the invention can be used to isolate corresponding sequences from other organisms, particularly other plants. In this manner, methods such as PCR, hybridization, and the like can be used to identify such sequences based on their sequence homology to the sequences set forth herein. Sequences isolated based on their sequence identity to the entire plant defense sequences set forth herein or to fragments thereof are encompassed by the present invention. Such sequences include sequences that are orthologs of the disclosed sequences. “Orthologs” are genes derived from a common ancestral gene and are found in different species as a result of speciation. Genes found in different species are considered orthologs when their nucleotide sequences and/or their encoded protein sequences share substantial identity as defined elsewhere herein. Functions of orthologs are often highly conserved among species. Thus, isolated sequences that encode a plant defense protein and which hybridize under stringent conditions to the plant defense sequences disclosed herein, or to fragments thereof, are encompassed by the present invention.

In a PCR approach, oligonucleotide primers can be designed for use in PCR reactions to amplify corresponding DNA sequences from cDNA or genomic DNA extracted from any plant of interest. Methods for designing PCR primers and PCR cloning are generally known in the art and are disclosed in Sambrook et al. (1989) Molecular Cloning: A Laboratory Manual (2d ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen organism. The hybridization probes may be genomic DNA fragments, cDNA fragments, RNA fragments, or other oligonucleotides, and may be labeled with a detectable group such as ³²P, or any other detectable marker. Thus, for example, probes for hybridization can be made by labeling synthetic oligonucleotides based on the plant defense sequences of the invention. Methods for preparation of probes for hybridization and for construction of cDNA and genomic libraries are generally known in the art and are disclosed in Sambrook et al. (1989) Molecular Cloning: A Laboratory Manual (2d ed., Cold Spring Harbor Laboratory Press, Plainview, N.Y.).

For example, an entire plant defense sequence disclosed herein, or one or more portions thereof, may be used as a probe capable of specifically hybridizing to corresponding plant defense sequences and messenger RNAs. To achieve specific hybridization under a variety of conditions, such probes include sequences that are unique among plant defense sequences and are preferably at least about 10 nucleotides in length, and most preferably at least about 20 nucleotides in length. Such probes may be used to amplify corresponding sequences from a chosen organism by PCR. This technique may be used to isolate additional coding sequences from a desired organism or as a diagnostic assay to determine the presence of coding sequences in an organism. Hybridization techniques include hybridization screening of plated DNA libraries (either plaques or colonies; see, for example, Sambrook et al. (1989) Molecular Cloning: A Laboratory Manual (2d ed., Cold Spring Harbor Laboratory Press, Plainview, N.Y.).

Hybridization of such sequences may be carried out under stringent conditions. “Stringent conditions” or “stringent hybridization conditions” are conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Optionally, wash buffers may comprise about 0.1% to about 1% SDS. Duration of hybridization is generally less than about 24 hours, usually about 4 to about 12 hours.

Specificity is typically the function of post-hybridization washes, the critical factors being the ionic strength and temperature of the final wash solution. For DNA-DNA hybrids, the thermal melting point (Tₘ) can be approximated from the equation of Meinkoth and Wahl (1984) Anal. Biochem. 138:267-284: Tₘ=81.5° C.+16.6 (log M)+0.41 (% GC)−0.61 (% form)−500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired Tₘ, those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, N.Y.).

Thus, isolated sequences that encode a plant defense polypeptide and which hybridize under stringent conditions to the plant defense sequences disclosed herein, or to fragments thereof, are encompassed by the present invention.

The following terms are used to describe the sequence relationships between two or more nucleic acids or polynucleotides: (a) “reference sequence”, (b) “comparison window”, (c) “sequence identity”, (d) “percentage of sequence identity”, and (e) “substantial identity”.

(a) As used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS 4:11-17; the local homology algorithm of Smith et al. (1981) Adv. Appl. Math. 2:482; the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; the algorithm of Karlin and Altschul (1990) Proc. Natl. Acad. Sci. USA 87:2264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al. (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See, for example, the website for the National Center for Biotechnology Information which can be found using an internet search engine. Alignment may also be performed manually by inspection.

Unless otherwise stated, sequence identity/similarity values provided herein refer to the value obtained using GAP Version 10 using the following parameters: % identity using GAP Weight of 50 and Length Weight of 3; % similarity using Gap Weight of 12 and Length Weight of 4, or any equivalent program. An “equivalent program” is any sequence comparison program that, for any two sequences in question, generates an alignment having identical nucleotide or amino acid residue matches and an identical percent sequence identity when compared to the corresponding alignment generated by the preferred program.

GAP uses the algorithm of Needleman and Wunsch (1970) J. Mol. Biol. 48:443-453, to find the alignment of two complete sequences that maximizes the number of matches and minimizes the number of gaps. GAP considers all possible alignments and gap positions and creates the alignment with the largest number of matched bases and the fewest gaps. It allows for the provision of a gap creation penalty and a gap extension penalty in units of matched bases. GAP must make a profit of gap creation penalty number of matches for each gap it inserts. If a gap extension penalty greater than zero is chosen, GAP must, in addition, make a profit for each gap inserted of the length of the gap times the gap extension penalty. Default gap creation penalty values and gap extension penalty values in Version 10 of the Wisconsin Genetics Software Package for protein sequences are 8 and 2, respectively. For nucleotide sequences the default gap creation penalty is 50 while the default gap extension penalty is 3. The gap creation and gap extension penalties can be expressed as an integer selected from the group of integers consisting of from 0 to 200. Thus, for example, the gap creation and gap extension penalties can be 0, 1, 2, 3, 4, 5, 6, 7, 8, 9, 10, 15, 20, 25, 30, 35, 40, 45, 50, 55, 60, 65 or greater.

GAP presents one member of the family of best alignments. There may be many members of this family, but no other member has a better quality. GAP displays four figures of merit for alignments: Quality, Ratio, Identity, and Similarity. The Quality is the metric maximized in order to align the sequences. Ratio is the quality divided by the number of bases in the shorter segment. Percent Identity is the percent of the symbols that actually match. Percent Similarity is the percent of the symbols that are similar. Symbols that are across from gaps are ignored. A similarity is scored when the scoring matrix value for a pair of symbols is greater than or equal to 0.50, the similarity threshold. The scoring matrix used in Version 10 of the Wisconsin Genetics Software Package is BLOSUM62 (see Henikoff and Henikoff (1989) Proc. Natl. Acad. Sci. USA 89:10915).

(c) As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

(d) As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

(e)(i) The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

Another indication that nucleotide sequences are substantially identical is if two molecules hybridize to each other under stringent conditions. Generally, stringent conditions are selected to be about 5° C. lower than the Tₘ for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

(e)(ii) The term “substantial identity” in the context of a peptide indicates that a peptide comprises a sequence with at least 70% sequence identity to a reference sequence, preferably 80%, more preferably 85%, most preferably at least 90% or 95% sequence identity to the reference sequence over a specified comparison window. Preferably, optimal alignment is conducted using the homology alignment algorithm of Needleman et al. (1970) J. Mol. Biol. 48:443. An indication that two peptide sequences are substantially identical is that one peptide is immunologically reactive with antibodies raised against the second peptide. Thus, a peptide is substantially identical to a second peptide, for example, where the two peptides differ only by a conservative substitution. Peptides that are “substantially similar” share sequences as noted above except that residue positions that are not identical may differ by conservative amino acid changes.

### Disease and Pests

Compositions and methods for controlling pathogenic agents are provided. The anti-pathogenic compositions comprise plant defense nucleotide and amino acid sequences. Particularly, the plant nucleic acid and amino acid sequences and fragments and variants thereof set forth herein possess anti-pathogenic activity. Accordingly, the compositions and methods are useful in protecting plants against fungal pathogens, nematodes, insects, and the like. Additionally provided are transformed plants, plant cells, plant tissues and seeds thereof.

A “plant pathogen” or “plant pest” is any organism that can cause harm to a plant, by inhibiting or slowing the growth of a plant, by damaging the tissues of a plant, by weakening the immune system of a plant, reducing the resistance of a plant to abiotic stresses, and/or by causing the premature death of the plant, etc. Plant pathogens and plant pests include insects, nematodes, and organisms such as fungi, and bacteria. “Disease resistance” or “pathogen resistance” means that the organisms avoid the disease symptoms which are the outcome of organism-pathogen interactions. That is, pathogens are prevented from causing diseases and the associated disease symptoms, or alternatively, the disease symptoms caused by the pathogen is minimized or lessened.

An “anti-pathogenic composition” is a composition of the invention that is capable of suppressing, controlling, and/or killing the invading pathogenic organism. An antipathogenic composition of the invention will reduce the disease symptoms resulting from pathogen challenge by at least about 5% to about 50%, at least about 10% to about 60%, at least about 30% to about 70%, at least about 40% to about 80%, or at least about 50% to about 90% or greater. Hence, the methods of the invention can be utilized to protect plants from disease, particularly those diseases that are caused by plant pathogens.

An “antimicrobial agent,” a “pesticidal agent,” an “insecticidal agent,” and/or a “fungicidal agent” will act similarly to suppress, control, and/or kill the invading pathogen.

Assays that measure antipathogenic activity are commonly known in the art, as are methods to quantitate disease resistance in plants following pathogen infection. See, for example, U.S. Pat. No. 5,614,395, herein incorporated by reference. Such techniques include, measuring over time, the average lesion diameter, the pathogen biomass, and the overall percentage of decayed plant tissues. For example, a plant either expressing an antipathogenic polypeptide or having an antipathogenic composition applied to its surface shows a decrease in tissue necrosis (i.e., lesion diameter) or a decrease in plant death following pathogen challenge when compared to a control plant that was not exposed to the antipathogenic composition. Alternatively, antipathogenic activity can be measured by a decrease in pathogen biomass. For example, a plant expressing an antipathogenic polypeptide or exposed to an antipathogenic composition is challenged with a pathogen of interest. Over time, tissue samples from the pathogen-inoculated tissues are obtained and RNA is extracted. The percent of a specific pathogen RNA transcript relative to the level of a plant specific transcript allows the level of pathogen biomass to be determined. See, for example, Thomma et al. (1998) Plant Biology 95:15107-15111, herein incorporated by reference.

Furthermore, in vitro antipathogenic assays include, for example, the addition of varying concentrations of the antipathogenic composition to paper disks and placing the disks on agar containing a suspension of the pathogen of interest. Following incubation, clear inhibition zones develop around the discs that contain an effective concentration of the antipathogenic polypeptide (Liu et al. (1994) Plant Biology 91:1888-1892, herein incorporated by reference). Additionally, microspectrophotometrical analysis can be used to measure the in vitro antipathogenic properties of a composition (Hu et al. (1997) Plant Mol. Biol. 34:949-959 and Cammue et al. (1992) J. Biol. Chem. 267: 2228-2233, both of which are herein incorporated by reference).

In specific embodiments, methods for increasing pathogen resistance in a plant comprise stably transforming a plant with a DNA construct comprising an anti-pathogenic nucleotide sequence of the invention operably linked to promoter that drives expression in a plant. Such methods find use in agriculture particularly in limiting the impact of plant pathogens on crop plants. While the choice of promoter will depend on the desired timing and location of expression of the anti-pathogenic nucleotide sequences, preferred promoters include constitutive and pathogen-inducible promoters.

It is understood in the art that plant DNA viruses and fungal pathogens remodel the control of the host replication and gene expression machinery to accomplish their own replication and effective infection. The present invention may be useful in preventing such corruption of the cell.

The plant defense sequences find use in disrupting cellular function of plant pathogens or insect pests as well as altering the defense mechanisms of a host plant to enhance resistance to disease or insect pests. While the invention is not bound by any particular mechanism of action to enhance disease resistance, the gene products of the plant defense sequences function to inhibit or prevent diseases in a plant.

The methods of the invention can be used with other methods available in the art for enhancing disease resistance in plants. For example, any one of a variety of second nucleotide sequences may be utilized, embodiments of the invention encompass those second nucleotide sequences that, when expressed in a plant, help to increase the resistance of a plant to pathogens. It is recognized that such second nucleotide sequences may be used in either the sense or antisense orientation depending on the desired outcome.

Pathogens of the invention include, but are not limited to, bacteria, insects, nematodes, fungi, and the like. Specific fungal, bacterial and viral pathogens affecting various major crops include, but are not limited to: Acremonium strictum; Albugo candida, A. tragopogonis; Alternaria alternate, A. brassicae, A. helianthi, A. zinniae; Aphanomyces euteiches; Ascochyta sorghina, A. tritici; Aspergillus flavus; Bipolaris maydis, B. sorghicola, B. sorokiniana; Botrytis cinerea; Cephalosporium acremonium, C. gramineum, C. maydis; Cercospora kikuchii, C. sorghi, C. medicaginis, C. sojina; Cladosporium herbarum; Clavibacter michiganense; Claviceps purpurea, C. sorghi; Cochliobolus carbonum, C. heterostrophus; Colletotrichum dematium (Colletotrichum truncatum), C. graminicola; Corynespora cassiicola; Curvularia inaequalis, C. lunata, C. pallescens; Diaporthe phaseolorum; Diplodia maydis, D. macrospora; Erwinia carotovorum, E. chrysanthemi, E. stewartii; Erysiphe cichoracearum, E. graminis; Exserohilum turcicum; Fusarium avenaceum, F. graminearum, F. culmorum, F. moniliforme, F. oxysporum, F. roseum, F. semitectum, F. solani; Gaeumannomyces graminis; Gloeocercospora sorghi; Glomerella glycines; Helminthosporium carbonum, H. sorghicola, H. pedicellatum; Kabatiella maydis; Leptosphaeria maculans; Leptotrochila medicaginis; Macrophomina phaseolina; Microsphaera diffusa; Mycosphaerella brassiccola; Nigrospora oryzae; Penicillium oxalicum; Periconia circinate; Peronosclerospora maydis, P. philippinensis, P. sacchari, P. sorghi; Peronospora manshurica, P. parasitica, P. trifoliorum; Phakopsora pachyrhizi; Phialophora gregata; Phoma insidiosa, P. macdonaldii, P. medicaginis; Phomopsis helianthi; Phyllachara sacchari; Phyllosticta maydis, P. sojicola; Physoderma maydis; Physopella zeae; Phytophthora cryptogea, P. megasperma; Plasmophora halstedii; Pseudocercosporella herpotrichoides; Pseudomonas andropogonis, P. avenae, P. syringae; Pseudopeziza medicaginis; Puccinia graminis, P. helianthi, P. polysora, P. purpurea, P. recondite, P. sorghi, P. striiformis; Pyrenophora tritici-repentis; Pythium aphanidermatum, P. arrhenomanes, P. debaryanum, P. graminicola, P. irregulare, P. splendens, P. ultimum; Ramulispora sorghi, R. sorghicola; Rhizoctonia cerealis, R. solani, Rhizopus arrhizus, R. oryzae, R. stolonifer; Sclerophthora macrospora; Sclerotinia sclerotiorum; Sclerotium rolfsii; Septoria avenae, S. glycines, S. helianthi, S. nodorum, S. tritici; Sphacelotheca reiliana, S. cruenta; Sporisorium reilianum, S. sorghi; Stemphylium alfalfae, S. herbarum; Tilletia indica, T. laevis, T. tritici; Trichoderma viride; Urocystis agropyri; Ustilago maydis, U. tritici; Verticillium dahliae; and Xanthomonas campestris.

Nematodes include parasitic nematodes such as root-knot, cyst, and lesion nematodes, including Heterodera and Globodera spp.; particularly Globodera rostochiensis and Globodera pailida (potato cyst nematodes); Heterodera glycines (soybean cyst nematode); Heterodera schachtii (beet cyst nematode); and Heterodera avenae (cereal cyst nematode). Additional nematodes include: Heterodera cajani, H. trifolii, H. oryzae; Globodera tabacum; Meloidogyne incognita, M. javonica, M. hapla, M. arenaria, M. naasi, M. exigua; Xiphinema index, X. italiae, X. americanum, X. diversicaudatum; Pratylenchus penetrans, P. brachyurus, P. zeae, P. coffeae, P. thornei, P. scribneri, P. vulnus, P. curvitatus; Radopholus similis, R. citrophilus; Ditylenchus angustus, D. dipsaci; Helicotylenchus multicintus; Rotylenchulus reniformis; Belonolaimus spp.; Paratrichodorus anemones; Trichodorus spp.; Primitivus spp.; Anguina tritici; Bider avenae; Subanguina radicicola; Tylenchorynchus spp.; Hoplolaimus seinhorsti; Tylenchulus semipenetrans; Hemicycliophora arenaria; Paratrichodorus xiphinema, P. minor, P. christiei; Rhadinaphelenchus cocophilus; Hoplolaimus galeatus, H. columbus; Criconemella spp.; Paratylenchus spp.; Nacoabbus aberrans; Aphelenchoides besseyi; Hirchmaniella spp.; Scutellonema spp.; Hemicriconemoides kanayaensis; and Cacopaurus pestis.

Those skilled in the art will recognize that not all compounds are equally effective against all pests. Insect pests include economically important agronomic, forest, greenhouse, nursery, ornamentals, food and fiber, public and animal health, domestic and commercial structure, household, and stored product pests. Insect pests include insects selected from the orders Coleoptera, Diptera, Hymenoptera, Lepidoptera, Mallophaga, Homoptera, Hemiptera, Orthoptera, Thysanoptera, Dermaptera, Isoptera, Anoplura, Siphonaptera, Trichoptera, etc., particularly Coleoptera and Lepidoptera.

Larvae of the order Lepidoptera include, but are not limited to, armyworms, cutworms, loopers, and heliothines in the family Noctuidae, such as Spodoptera frugiperda, S. exigua, S. litura; Mamestra configurata, M. brassicae; Agrotis ipsilon, A. orthogonia, A. subterranea; Alabama argillacea; Trichoplusia ni; Pseudoplusia includens; Anticarsia gemmatalis; Hypena scabs; Heliothis virescens; Pseudaletia unipuncta; Athetis mindara; Euxoa messoria; Earias insulana, E. vittella; Helicoverpa armigera, H. zea; Melanchra picta; Egira (Xylomyges) curialis; borers, casebearers, webworms, coneworms, and skeletonizers from the family Pyralidae, such as Ostrinia nubilali; Amyelois transitella; Anagasta kuehniella; Cadra cautella; Chilo suppressalis, C. partellus; Corcyra cephalonica; Crambus caliginosellus, C. teterrellus; Cnaphalocrocis medinalis; Desmia funeralis; Diaphania hyalinata, D. nitidalis; Diatraea grandiosella, D. saccharali; Eoreuma loftini; Ephestia elutella; Galleria mellonella; Herpetogramma licarsisalis; Homoeosoma electellum; Elasmopalpus lignosellus; Achroia grisella; Loxostege sticticali; Orthaga thyrisalis; Maruca testulalis; Plodia interpunctella; Scirpophaga incertula; Udea rubigalis; and leafrollers, budworms, seed worms, and fruit worms in the family Tortricidae, such as Acleris gloverana, A. variana; Archips argyrospila, A. rosana; and other Archips species, Adoxophyes orana; Cochylis hospes; Cydia latiferreana, C. pomonella; Platynota flavedana, P. stultana; Lobesia botrana; Spilonota ocellana; Endopiza viteana; Eupoecilia ambiguella; Bonagota salubricola; Grapholita molesta; Suleima helianthana; Argyrotaenia spp.; Choristoneura spp.

Selected other agronomic pests in the order Lepidoptera include, but are not limited to, Alsophila pometaria; Anarsia lineatella; Anisota senatoria; Antheraea pernyi; Bombyx mor; Bucculatrix thurberiella; Colias eurytheme; Datana integerrima; Dendrolimus sibiricus; Ennomos subsignaria; Erannis tiliaria; Euproctis chrysorrhoea; Harrisina americana; Hemileuca oliviae; Hyphantria cunea; Keiferia lycopersicella; Lambdina fiscellaria fiscellaria, L. fiscellaria lugubrosa; Leucoma salicis; Lymantria dispar; Manduca quinquemaculata, M. sexta; Operophtera brumata; Paleacrita vernata; Papilio cresphontes; Phryganidia califormica; Phyllocnistis citrella; Phyllonorycter blancardella; Pieris brassicae, P. rapae, P. napi; Platyptilia carduidactyla; Plutella xylostella; Pectinophora gossypiella; Pontia protodice; Sabulodes aegrotata; Schizura concinna; Sitotroga cerealella; Thaumetopoea pityocampa; Tineola bisselliella; Tuta absoluta; Yponomeuta padella; Heliothis subflexa; Malacosoma spp. and Orgyia spp.

Of interest are larvae and adults of the order Coleoptera including weevils from the families Anthribidae, Bruchidae, and Curculionidae, including, but not limited to: Anthonomus grandis; Lissorhoptrus oryzophilus; Sitophilus granarius, S. oryzae; Hypera punctata; Cylindrocopturus adspersus; Smicronyx fulvus, S. sordidu; Sphenophorus maidi; flea beetles, cucumber beetles, rootworms, leaf beetles, potato beetles, and leafminers in the family Chrysomelidae including, but not limited to: Leptinotarsa decemlineata; Diabrotica virgifera virgifera, D. barberi, D. undecimpunctata howardi; Chaetocnema pulicaria; Phyllotreta cruciferae; Colaspis brunnea; Oulema melanopus; Zygogramma exclamationis; beetles from the family Coccinellidae including, but not limited to: Epilachna varivestis; chafers and other beetles from the family Scarabaeidae including, but not limited to: Popillia japonica; Cyclocephala borealis, C. immaculata; Rhizotrogus majalis; Phyllophaga crinita; Ligyrus gibbosus; carpet beetles from the family Dermestidae; wireworms from the family Elateridae, Eleodes spp., Melanotus spp.; Conoderus spp.; Limonius spp.; Agriotes spp.; Ctenicera spp.; Aeolus spp.; bark beetles from the family Scolytidae and beetles from the family Tenebrionidae.

Adults and immatures of the order Diptera are of interest, including leafminers Agromyza parvicornis; midges (including, but not limited to: Contarinia sorghicola; Mayetiola destructor; Sitodiplosis mosellana; Neolasioptera murtfeldtiana; fruit flies (Tephritidae), Oscinella frit; maggots including, but not limited to: Delia platura, D. coarctate; and other Delia spp., Meromyza americana; Musca domestica; Fannia canicularis, F. femoralis; Stomoxys calcitrans; face flies, horn flies, blow flies, Chrysomya spp.; Phormia spp.; and other muscoid fly pests, horse flies Tabanus spp.; bot flies Gastrophilus spp.; Oestrus spp.; cattle grubs Hypoderma spp.; deer flies Chrysops spp.; Melophagus ovinus and other Brachycera, mosquitoes Aedes spp.; Anopheles spp.; Culex spp.; black flies Prosimulium spp.; Simulium spp.; biting midges, sand flies, sciarids, and other Nematocera.

Selected other agronomic pests in the order Lepidoptera include, but are not limited to, Alsophila pometaria; Anarsia lineatella; Anisota senatoria; Antheraea pernyi; Bombyx mori; Bucculatrix thurberiella; Colias eurytheme; Datana integerrima; Dendrolimus sibiricus; Ennomos subsignaria; Erannis tiliaria; Euproctis chrysorrhoea; Harrisina americana; Hemileuca oliviae; Hyphantria cunea; Keiferia lycopersicella; Lambdina fiscellaria fiscellaria, L. fiscellaria lugubrosa; Leucoma salicis; Lymantria dispar; Manduca quinquemaculata, M. sexta; Operophtera brumata; Paleacrita vernata; Papilio cresphontes; Phryganidia califormica; Phyllocnistis citrella; Phyllonorycter blancardella; Pieris brassicae, P. rapae, P. napi; Platyptilia carduidactyla; Plutella xylostella; Pectinophora gossypiella; Pontia protodice; Sabulodes aegrotata; Schizura concinna; Sitotroga cerealella; Thaumetopoea pityocampa; Tineola bisselliella; Tuta absoluta; Yponomeuta padella; Heliothis subflexa; Malacosoma spp. and Orgyia spp.

Included as insects of interest are adults and nymphs of the orders Hemiptera and Homoptera such as, but not limited to, adelgids from the family Adelgidae, plant bugs from the family Miridae, cicadas from the family Cicadidae, leafhoppers, Empoasca spp.; from the family Cicadellidae, planthoppers from the families Cixiidae, Flatidae, Fulgoroidea, Issidae and Delphacidae, treehoppers from the family Membracidae, psyllids from the family Psyllidae, whiteflies from the family Aleyrodidae, aphids from the family Aphididae, phylloxera from the family Phylloxeridae, mealybugs from the family Pseudococcidae, scales from the families Asterolecanidae, Coccidae, Dactylopiidae, Diaspididae, Eriococcidae, Ortheziidae, Phoenicococcidae and Margarodidae, lace bugs from the family Tingidae, stink bugs from the family Pentatomidae, cinch bugs, Blissus spp.; and other seed bugs from the family Lygaeidae, spittlebugs from the family Cercopidae squash bugs from the family Coreidae, and red bugs and cotton stainers from the family Pyrrhocoridae.

Agronomically important members from the order Homoptera further include, but are not limited to: Acyrthisiphon pisum; Aphis craccivora, A. fabae, A. gossypi, A. maidiradicis, A. pomi, A. spiraecola; Aulacorthum solani; Chaetosiphon fragaefolii; Diuraphis noxia; Dysaphis plantaginea; Eriosoma lanigerum; Brevicoryne brassicae; Hyalopterus pruni; Lipaphis erysimi; Metopolophium dirrhodum; Macrosiphum euphorbiae; Myzus persicae; Nasonovia ribisnigri; Pemphigus spp.; Rhopalosiphum maidis, R. padi; Schizaphis graminum; Sipha flava; Sitobion avenae; Therioaphis maculata; Toxoptera aurantii and T. citricida; Adelges spp. (adelgids); Phylloxera devastatrix; Bemisia tabaci, B. argentifolii; Dialeurodes citri; Trialeurodes abutiloneus, T. vaporariorum; Empoasca fabae; Laodelphax striatellus; Macrolestes quadrilineatus; Nephotettix cinticeps, N. nigropictus; Nilaparvata lugens; Peregrinus maidis; Sogatella furcifera; Sogatodes orizicola; Typhlocyba pomaria; Erythroneoura spp.; Magicicada septendecim; Icerya purchasi; Quadraspidiotus perniciosus; Planococcus citri; Pseudococcus spp.; Cacopsylla pyricola; Trioza diospyri.

Agronomically important species of interest from the order Hemiptera include, but are not limited to: Acrosternum hilare; Adelphocoris rapidus; Anasa tristis; Blissus leucopterus leucopterus; Calocoris norvegicus; Corythuca gossypii; Cyrtopeltis modesta, C. notatus; Diaphnocoris chlorionis; Dysdercus suturellus; Euschistus servus, E. variolarius; Graptostethus spp.; Labopidicola allii; Leptoglossus corculus; Lygus lineolari, L. Hesperus, L. pratensis, L. rugulipennis; Lygocoris pabulinus; Nezara viridula; Nysius ericae, N. raphanus; Oebalus pugnax; Oncopeltus fasciatus; Orthops campestris; Plesiocoris rugicollis; Poecilocapsus lineatus; Pseudatomoscelis seriatus; Spanagonicus albofasciatus; Eurygaster spp.; Coreidae spp.; Pyrrhocoridae spp.; Timidae spp.; Blostomatidae spp.; Reduviidae spp. and Cimicidae spp.

Adults and immatures of the insect order Orthoptera are of interest, including grasshoppers, locusts and crickets Melanoplus sanguinipes, M. differentialis, M. femurrubrum; Schistocerca americana, S. gregaria; Locusta migratoria; Acheta domesticus; and Gryllotalpa spp.

Adults and larvae of the order Thysanoptera are of interest, including Thrips tabaci; Anaphothrips obscrurus; Frankliniella fusca, F. occidentalis; Neohydatothrips variabilis; Scirthothrips citri and other foliar feeding thrips.

Also included are adults and larvae of the order Acari (mites) such as Aceria tosichella; Petrobia latens; spider mites and red mites in the family Tetranychidae, Panonychus ulmi; Tetranychus urtica; T. mcdanieli, T. cinnabarinus, T. turkestani; flat mites in the family Tenuipalpidae, Brevipalpus lewisi; rust and bud mites in the family Eriophyidae and other foliar feeding mites and mites important in human and animal health, i.e. dust mites in the family Epidermoptidae, follicle mites in the family Demodicidae, grain mites in the family Glycyphagidae, ticks in the order Ixodidae. Ixodes scapularis, I. holocyclus; Dermacentor variabilis; Amblyomma americanum; and scab and itch mites in the families Psoroptidae, Pyemotidae, and Sarcoptidae.

Insect pests of the order Thysanura are of interest, such as Lepisma saccharina and Thermobia domestica.

Additional arthropod pests covered include: spiders in the order Araneae such as Loxosceles reclusa; and Latrodectus mactans; and centipedes in the order Scutigeromorpha such as Scutigera coleoptrata.

### Expression of Sequences

The nucleic acid sequences of the present invention can be expressed in a host cell such as bacterial, fungal, yeast, insect, mammalian, or preferably plant cells. It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous nucleotide sequence can be from a species different from that from which the nucleotide sequence was derived, or, if from the same species, the promoter is not naturally found operably linked to the nucleotide sequence. A heterologous protein may originate from a foreign species, or, if from the same species, is substantially modified from its original form by deliberate human intervention.

By “host cell” a cell, which comprises a heterologous nucleic acid sequence of the invention is meant. Host cells may be prokaryotic cells such as E. coli, or eukaryotic cells such as yeast, insect, amphibian, or mammalian cells. Preferably, host cells are monocotyledonous or dicotyledonous plant cells. A particularly preferred monocotyledonous host cell is a maize host cell.

The plant defense sequences of the invention are provided in expression cassettes or DNA constructs for expression in the plant of interest. The cassette will include 5′ and 3′ regulatory sequences operably linked to a plant defense sequence of the invention. “Operably linked” means that there is a functional linkage between a promoter and a second sequence, wherein the promoter sequence initiates and mediates transcription of the DNA sequence corresponding to the second sequence. Generally, operably linked means that the nucleic acid sequences being linked are contiguous and, where necessary to join two protein coding regions, contiguous and in the same reading frame. The cassette may additionally contain at least one additional gene to be cotransformed into the organism. Alternatively, the additional gene(s) can be provided on multiple expression cassettes.

Such an expression cassette is provided with a plurality of restriction sites for insertion of the plant defense sequence to be under the transcriptional regulation of the regulatory regions. The expression cassette may additionally contain selectable marker genes.

The expression cassette will include in the 5′-3′ direction of transcription, a transcriptional and translational initiation region, a plant defense DNA sequence of the invention, and a transcriptional and translational termination region functional in plants. The transcriptional initiation region, the promoter, may be native or analogous or foreign or heterologous to the plant host. Additionally, the promoter may be the natural sequence or alternatively a synthetic sequence. “Foreign” means that the transcriptional initiation region is not found in the native plant into which the transcriptional initiation region is introduced. As used herein, a chimeric gene comprises a coding sequence operably linked to a transcription initiation region that is heterologous to the coding sequence.

While it may be preferable to express the sequences using heterologous promoters, the native promoter sequences may be used. Such constructs would change expression levels of plant defense proteins in the host cell (i.e., plant or plant cell). Thus, the phenotype of the host cell (i.e., plant or plant cell) is altered.

The termination region may be native with the transcriptional initiation region, may be native with the operably linked DNA sequence of interest, or may be derived from another source. Convenient termination regions are available from the Ti-plasmid of A. tumefaciens, such as the octopine synthase and nopaline synthase termination regions. See also Guerineau et al. (1991) Mol. Gen. Genet. 262:141-144; Proudfoot (1991) Cell 64:671-674; Sanfacon et al. (1991) Genes Dev. 5:141-149; Mogen et al. (1990) Plant Cell 2:1261-1272; Munroe et al. (1990) Gene 91:151-158; Ballas et al. (1989) Nucleic Acids Res. 17:7891-7903; and Joshi et al. (1987) Nucleic Acid Res. 15:9627-9639.

Where appropriate, the gene(s) may be optimized for increased expression in the transformed plant. That is, the genes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, 5,436,391, and Murray et al. (1989) Nucleic Acids Res. 17:477-498, herein incorporated by reference.

Additional sequence modifications are known to enhance gene expression in a cellular host. These include elimination of sequences encoding spurious polyadenylation signals, exon-intron splice site signals, transposon-like repeats, and other such well-characterized sequences that may be deleterious to gene expression. The G-C content of the sequence may be adjusted to levels average for a given cellular host, as calculated by reference to known genes expressed in the host cell. When possible, the sequence is modified to avoid predicted hairpin secondary mRNA structures.

The expression cassettes may additionally contain 5′ leader sequences in the expression cassette construct. Such leader sequences can act to enhance translation. Translation leaders are known in the art and include: picornavirus leaders, for example, EMCV leader (Encephalomyocarditis 5′ noncoding region) (Elroy-Stein et al. (1989) PNAS USA 86:6126-6130); potyvirus leaders, for example, TEV leader (Tobacco Etch Virus) (Allison et al. (1986); MDMV leader (Maize Dwarf Mosaic Virus); Virology 154:9-20), and human immunoglobulin heavy-chain binding protein (BiP), (Macejak et al. (1991) Nature 353:90-94); untranslated leader from the coat protein mRNA of alfalfa mosaic virus (AMV RNA 4) (Jobling et al. (1987) Nature 325:622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968. Other methods known to enhance translation can also be utilized, for example, introns, and the like.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

Generally, the expression cassette will comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glyphosate, glufosinate ammonium, bromoxynil, imidazolinones, and 2,4-dichlorophenoxyacetate (2,4-D). See generally, Yarranton (1992) Curr. Opin. Biotech. 3:506-511; Christopherson et al. (1992) Proc. Natl. Acad. Sci. USA 89:6314-6318; Yao et al. (1992) Cell 71:63-72; Reznikoff (1992) Mol. Microbiol. 6:2419-2422; Barkley et al. (1980) in The Operon, pp. 177-220; Hu et al. (1987) Cell 48:555-566; Brown et al. (1987) Cell 49:603-612; Figge et al. (1988) Cell 52:713-722; Deuschle et al. (1989) Proc. Natl. Acad. Sci. USA 86:5400-5404; Fuerst et al. (1989) Proc. Natl. Acad. Sci. USA 86:2549-2553; Deuschle et al. (1990) Science 248:480-483; Gossen (1993) Ph.D. Thesis, University of Heidelberg; Reines et al. (1993) Proc. Natl. Acad. Sci. USA 90:1917-1921; Labow et al. (1990) Mol. Cell. Biol. 10:3343-3356; Zambretti et al. (1992) Proc. Natl. Acad. Sci. USA 89:3952-3956; Baim et al. (1991) Proc. Natl. Acad. Sci. USA 88:5072-5076; Wyborski et al. (1991) Nucleic Acids Res. 19:4647-4653; Hillenand-Wissman (1989) Topics Mol. Struc. Biol. 10:143-162; Degenkolb et al. (1991) Antimicrob. Agents Chemother. 35:1591-1595; Kleinschnidt et al. (1988) Biochemistry 27:1094-1104; Bonin (1993) Ph.D. Thesis, University of Heidelberg; Gossen et al. (1992) Proc. Natl. Acad. Sci. USA 89:5547-5551; Oliva et al. (1992) Antimicrob. Agents Chemother. 36:913-919; Hlavka et al. (1985) Handbook of Experimental Pharmacology, Vol. 78 (Springer-Verlag, Berlin); Gill et al. (1988) Nature 334:721-724. Such disclosures are herein incorporated by reference.

The above list of selectable marker genes is not meant to be limiting. Any selectable marker gene can be used in the present invention.

A number of promoters can be used in the practice of the invention. The promoters can be selected based on the desired outcome. That is, the nucleic acids can be combined with constitutive, tissue-preferred, or other promoters for expression in the host cell of interest. Such constitutive promoters include, for example, the core promoter of the Rsyn7 promoter and other constitutive promoters disclosed in WO 99/43838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theon. Appl. Genet. 81:581-588); MAS (Velten et al. (1984) EMBO J. 3:2723-2730); ALS promoter (U.S. Pat. No. 5,659,026), and the like. Other constitutive promoters include, for example, those disclosed in U.S. Pat. Nos. 5,608,149; 5,608,144; 5,604,121; 5,569,597; 5,466,785; 5,399,680; 5,268,463; 5,608,142; and 6,177,611, herein incorporated by reference.

Generally, it will be beneficial to express the gene from an inducible promoter, particularly from a pathogen-inducible promoter. Such promoters include those from pathogenesis-related proteins (PR proteins), which are induced following infection by a pathogen; e.g., PR proteins, SAR proteins, beta-1,3-glucanase, chitinase, etc. See, for example, Redolfi et al. (1983) Neth. J. Plant Pathol. 89:245-254; Uknes et al. (1992) Plant Cell 4:645-656; and Van Loon (1985) Plant Mol. Virol. 4:111-116. See also WO 99/43819 published Sep. 9, 1999, herein incorporated by reference.

Of interest are promoters that are expressed locally at or near the site of pathogen infection. See, for example, Marineau et al. (1987) Plant Mol. Biol. 9:335-342; Matton et al. (1989) Molecular Plant-Microbe Interactions 2:325-331; Somsisch et al. (1986) Proc. Natl. Acad. Sci. USA 83:2427-2430; Somsisch et al. (1988) Mol. Gen. Genet. 2:93-98; and Yang (1996) Proc. Natl. Acad. Sci. USA 93:14972-14977. See also, Chen et al. (1996) Plant J. 10:955-966; Zhang et al. (1994) Proc. Natl. Acad. Sci. USA 91:2507-2511; Warner et al. (1993) Plant J. 3:191-201; Siebertz et al. (1989) Plant Cell 1:961-968; U.S. Pat. No. 5,750,386 (nematode-inducible); and the references cited therein. Of particular interest is the inducible promoter for the maize PRms gene, whose expression is induced by the pathogen Fusarium moniliforme (see, for example, Cordero et al. (1992) Physiol. Mol. Plant. Path. 41:189-200).

Additionally, as pathogens find entry into plants through wounds or insect damage, a wound-inducible promoter may be used in the constructions of the invention. Such wound-inducible promoters include potato proteinase inhibitor (pin II) gene (Ryan (1990) Ann. Rev. Phytopath. 28:425-449; Duan et al. (1996) Nature Biotechnology 14:494-498); wun1 and wun2, U.S. Pat. No. 5,428,148; win1 and win2 (Stanford et al. (1989) Mol. Gen. Genet. 215:200-208); systemin (McGurl et al. (1992) Science 225:1570-1573); WIP1 (Rohmeier et al. (1993) Plant Mol. Biol. 22:783-792; Eckelkamp et al. (1993) FEBS Letters 323:73-76); MPI gene (Corderok et al. (1994) Plant J. 6(2):141-150); and the like, herein incorporated by reference.

Chemical-regulated promoters can be used to modulate the expression of a gene in a plant through the application of an exogenous chemical regulator. Depending upon the objective, the promoter may be a chemical-inducible promoter, where application of the chemical induces gene expression, or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1a promoter, which is activated by salicylic acid. Other chemical-regulated promoters of interest include steroid-responsive promoters (see, for example, the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-preferred promoters can be utilized to target enhanced plant defense expression within a particular plant tissue. Tissue-preferred promoters include Yamamoto et al. (1997) Plant J. 12(2):255-265; Kawamata et al. (1997) Plant Cell Physiol. 38(7):792-803; Hansen et al. (1997) Mol. Gen. Genet. 254(3):337-343; Russell et al. (1997) Transgenic Res. 6(2):157-168; Rinehart et al. (1996) Plant Physiol. 112(3):1331-1341; Van Camp et al. (1996) Plant Physiol. 112(2):525-535; Canevascini et al. (1996) Plant Physiol. 112(2):513-524; Yamamoto et al. (1994) Plant Cell Physiol. 35(5):773-778; Lam (1994) Results Probl. Cell Differ. 20:181-196; Orozco et al. (1993) Plant Mol. Biol. 23(6):1129-1138; Matsuoka et al. (1993) Proc Natl. Acad. Sci. USA 90(20):9586-9590; and Guevara-Garcia et al. (1993) Plant J. 4(3):495-505. Such promoters can be modified, if necessary, for weak expression.

Leaf-specific promoters are known in the art. See, for example, Yamamoto et al. (1997) Plant J. 12(2):255-265; Kwon et al. (1994) Plant Physiol. 105:357-67; Yamamoto et al. (1994) Plant Cell Physiol. 35(5):773-778; Gotor et al. (1993) Plant J. 3:509-18; Orozco et al. (1993) Plant Mol. Biol. 23(6):1129-1138; and Matsuoka et al. (1993) Proc. Natl. Acad. Sci. USA 90(20):9586-9590.

“Seed-preferred” promoters include both “seed-specific” promoters (those promoters active during seed development such as promoters of seed storage proteins) as well as “seed-germinating” promoters (those promoters active during seed germination). See Thompson et al. (1989) BioEssays 10:108, herein incorporated by reference. Such seed-preferred promoters include, but are not limited to, Cim1 (cytokinin-induced message); cZ19B1 (maize 19 kDa zein); milps (myo-inositol-1-phosphate synthase); and celA (cellulose synthase) (see WO 00/11177, herein incorporated by reference). Gama-zein is a preferred endosperm-specific promoter. Glob-1 is a preferred embryo-specific promoter. For dicots, seed-specific promoters include, but are not limited to, bean β-phaseolin, napin, β-conglycinin, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also WO 00/12733, where seed-preferred promoters from end1 and end2 genes are disclosed; herein incorporated by reference.

The method of transformation/transfection is not critical to the instant invention; various methods of transformation or transfection are currently available. As newer methods are available to transform crops or other host cells they may be directly applied. Accordingly, a wide variety of methods have been developed to insert a DNA sequence into the genome of a host cell to obtain the transcription and/or translation of the sequence to effect phenotypic changes in the organism. Thus, any method, which provides for effective transformation/transfection may be employed.

Transformation protocols as well as protocols for introducing nucleotide sequences into plants may vary depending on the type of plant or plant cell, i.e., monocot or dicot, targeted for transformation. Suitable methods of introducing nucleotide sequences into plant cells and subsequent insertion into the plant genome include microinjection (Crossway et al. (1986) Biotechniques 4:320-334), electroporation (Riggs et al. (1986) Proc. Natl. Acad. Sci. USA 83:5602-5606, Agrobacterium-mediated transformation (Townsend et al., U.S. Pat. No. 5,563,055; Zhao et al., U.S. Pat. No. 5,981,840), direct gene transfer (Paszkowski et al. (1984) EMBO J. 3:2717-2722), and ballistic particle acceleration (see, for example, Sanford et al., U.S. Pat. No. 4,945,050; Tomes et al., U.S. Pat. No. 5,879,918; Tomes et al., U.S. Pat. No. 5,886,244; Bidney et al., U.S. Pat. No. 5,932,782; McCabe et al. (1988) Biotechnology 6:923-926); and Lec1 transformation (WO 00/28058). Also see Weissinger et al. (1988) Ann. Rev. Genet. 22:421-477; Sanford et al. (1987) Particulate Science and Technology 5:27-37 (onion); Christou et al. (1988) Plant Physiol. 87:671-674 (soybean); McCabe et al. (1988) Bio/Technology 6:923-926 (soybean); Finer and McMullen (1991) In Vitro Cell Dev. Biol. 27P:175-182 (soybean); Singh et al. (1998) Theor. Appl. Genet. 96:319-324 (soybean); Datta et al. (1990) Biotechnology 8:736-740 (rice); Klein et al. (1988) Proc. Natl. Acad. Sci. USA 85:4305-4309 (maize); Klein et al. (1988) Biotechnology 6:559-563 (maize); Tomes, U.S. Pat. No. 5,240,855; Buising et al., U.S. Pat. Nos. 5,322,783 and 5,324,646; Tomes et al. (1995) “Direct DNA Transfer into Intact Plant Cells via Microprojectile Bombardment,” in Plant Cell, Tissue, and Organ Culture: Fundamental Methods, ed. Gamborg (Springer-Verlag, Berlin) (maize); Klein et al. (1988) Plant Physiol. 91:440-444 (maize); Fromm et al. (1990) Biotechnology 8:833-839 (maize); Hooykaas-Van Slogteren et al. (1984) Nature (London) 311:763-764; Bowen et al., U.S. Pat. No. 5,736,369 (cereals); Bytebier et al. (1987) Proc. Natl. Acad. Sci. USA 84:5345-5349 (Liliaceae); De Wet et al. (1985) in The Experimental Manipulation of Ovule Tissues, ed. Chapman et al. (Longman, New York), pp. 197-209 (pollen); Kaeppler et al. (1990) Plant Cell Reports 9:415-418 and Kaeppler et al. (1992) Theor. Appl. Genet. 84:560-566 (whisker-mediated transformation); D′Halluin et al. (1992) Plant Cell 4:1495-1505 (electroporation); Li et al. (1993) Plant Cell Reports 12:250-255 and Christou and Ford (1995) Annals of Botany 75:407-413 (rice); Osjoda et al. (1996) Nature Biotechnology 14:745-750 (maize via Agrobacterium tumefaciens); all of which are herein incorporated by reference.

The cells that have been transformed may be grown into plants in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure that expression of the desired phenotypic characteristic has been achieved.

The present invention may be used for transformation of any plant species, including, but not limited to, monocots and dicots. Examples of plants of interest include, but are not limited to, corn (Zea mays), Brassica sp. (e.g., B. napus, B. rapa, B. juncea), particularly those Brassica species useful as sources of seed oil, alfalfa (Medicago sativa), rice (Oryza sativa), rye (Secale cereale), sorghum (Sorghum bicolor, S. vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum), soybean (Glycine max), tobacco (Nicotiana tabacum), potato (Solanum tuberosum), peanuts (Arachis hypogaea), cotton (Gossypium barbadense, G. hirsutum), sweet potato (Ipomoea batatas), cassaya (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Peryea americana), fig (Ficus spp.), guava (Psidium guajava), mango (Mangifera indica), olive (Olea europaea), papaya (Carica papaya), cashew (Anacardium occidentale), Macadamia (Macadamia integrifolia), almond (Prunus amygdalus), sugar beets (Beta vulgaris), sugarcane (Saccharum spp.), oats, barley, vegetables, ornamentals, and conifers.

Vegetables include tomatoes (Lycopersicon esculentum), lettuce (e.g., Lactuca sativa), green beans (Phaseolus vulgaris), lima beans (Phaseolus limensis), peas (Lathyrus spp., Pisum spp.), and members of the genus Cucumis such as cucumber (C. sativus), cantaloupe (C. cantalupensis), and musk melon (C. melo). Ornamentals include azalea (Rhododendron spp.), hydrangea (Hydrangea macrophylla), hibiscus (Hibiscus rosasanensis), roses (Rosa spp.), tulips (Tulipa spp.), daffodils (Narcissus spp.), petunias (Petunia hybrida), carnation (Dianthus caryophyllus), poinsettia (Euphorbia pulcherrima), and chrysanthemum. Conifers that may be employed in practicing the present invention include, for example, pines such as loblolly pine (Pinus taeda), slash pine (P. elliotii), ponderosa pine (P. ponderosa), lodgepole pine (P. contorta), and Monterey pine (P. radiata); Douglas-fir (Pseudotsuga menziesii); Western hemlock (Tsuga canadensis); Sitka spruce (Picea glauca); redwood (Sequoia sempervirens); true firs such as silver fir (Abies amabilis) and balsam fir (Abies balsamea); and cedars such as Western red cedar (Thuja plicata) and Alaska yellow-cedar (Chamaecyparis nootkatensis). Preferably, plants of the present invention are crop plants (for example, corn, alfalfa, sunflower, Brassica, soybean, cotton, safflower, peanut, sorghum, wheat, millet, tobacco, etc.), more preferably corn and soybean plants, yet more preferably corn plants.

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding sequences, include such commonly used promoters as the beta lactamase (penicillinase) and lactose (lac) promoter systems (Chang et al. (1977) Nature 198:1056), the tryptophan (trp) promoter system (Goeddel et al. (1980) Nucleic Acids Res. 8:4057) and the lambda derived PL promoter and N-gene ribosome binding site (Simatake and Rosenberg (1981) Nature 292:128). Examples of selection markers for E. coli include, for example, genes specifying resistance to ampicillin, tetracycline, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transfected with the plasmid vector DNA. Expression systems for expressing a protein of the present invention are available using Bacillus sp. and Salmonella (Palva et al. (1983) Gene 22:229-235 and Mosbach et al. (1983) Nature 302:543-545).

A variety of eukaryotic expression systems such as yeast, insect cell lines, plant and mammalian cells, are known to those of skill in the art. As explained briefly below, a polynucleotide of the present invention can be expressed in these eukaryotic systems. In some embodiments, transformed/transfected plant cells, as discussed infra, are employed as expression systems for production of the proteins of the instant invention. Such antimicrobial proteins can be used for any application including coating surfaces to target microbes as described further infra.

Synthesis of heterologous nucleotide sequences in yeast is well known. Sherman, F., et al. (1982) Methods in Yeast Genetics, Cold Spring Harbor Laboratory is a well recognized work describing the various methods available to produce proteins in yeast. Two widely utilized yeasts for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like, as desired.

A protein of the present invention, once expressed, can be isolated from yeast by lysing the cells and applying standard protein isolation techniques to the lysates. The monitoring of the purification process can be accomplished by using Western blot techniques, radioimmunoassay, or other standard immunoassay techniques.

The sequences of the present invention can also be ligated to various expression vectors for use in transfecting cell cultures of, for instance, mammalian, insect, or plant origin. Illustrative cell cultures useful for the production of the peptides are mammalian cells. A number of suitable host cell lines capable of expressing intact proteins have been developed in the art, and include the HEK293, BHK21, and CHO cell lines. Expression vectors for these cells can include expression control sequences, such as an origin of replication, a promoter (e.g. the CMV promoter, a HSV tk promoter or pgk (phosphoglycerate kinase) promoter), an enhancer (Queen et al. (1986) Immunol. Rev. 89:49), and necessary processing information sites, such as ribosome binding sites, RNA splice sites, polyadenylation sites (e.g., an SV40 large T Ag poly A addition site), and transcriptional terminator sequences. Other animal cells useful for production of proteins of the present invention are available, for instance, from the American Type Culture Collection.

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See, Schneider (1987) J. Embryol. Exp. Morphol. 27:353-365).

As with yeast, when higher animal or plant host cells are employed, polyadenylation or transcription terminator sequences are typically incorporated into the vector. An example of a terminator sequence is the polyadenylation sequence from the bovine growth hormone gene. Sequences for accurate splicing of the transcript may also be included. An example of a splicing sequence is the VP1 intron from SV40 (Sprague, et al. (1983) J. Virol. 45:773-781). Additionally, gene sequences to control replication in the host cell may be incorporated into the vector such as those found in bovine papilloma virus type-vectors. Saveria-Campo, M., (1985) Bovine Papilloma Virus DNA a Eukaryotic Cloning Vector in DNA Cloning Vol. II a Practical Approach, D. M. Glover, Ed., IRL Press, Arlington, Va. pp. 213-238.

Animal and lower eukaryotic (e.g., yeast) host cells are competent or rendered competent for transfection by various means. There are several well-known methods of introducing DNA into animal cells. These include: calcium phosphate precipitation, fusion of the recipient cells with bacterial protoplasts containing the DNA, treatment of the recipient cells with liposomes containing the DNA, DEAE dextrin, electroporation, biolistics, and micro-injection of the DNA directly into the cells. The transfected cells are cultured by means well known in the art. Kuchler, R. J. (1997) Biochemical Methods in Cell Culture and Virology, Dowden, Hutchinson and Ross, Inc.

It is recognized that with these nucleotide sequences, antisense constructions, complementary to at least a portion of the messenger RNA (mRNA) for the plant defense sequences can be constructed. Antisense nucleotides are constructed to hybridize with the corresponding mRNA. Modifications of the antisense sequences may be made as long as the sequences hybridize to and interfere with expression of the corresponding mRNA. In this manner, antisense constructions having 70%, preferably 80%, more preferably 85% sequence identity to the corresponding antisensed sequences may be used. Furthermore, portions of the antisense nucleotides may be used to disrupt the expression of the target gene. Generally, sequences of at least 50 nucleotides, 100 nucleotides, 200 nucleotides, or greater may be used.

The nucleotide sequences of the present invention may also be used in the sense orientation to suppress the expression of endogenous genes in plants. Methods for suppressing gene expression in plants using nucleotide sequences in the sense orientation are known in the art. The methods generally involve transforming plants with a DNA construct comprising a promoter that drives expression in a plant operably linked to at least a portion of a nucleotide sequence that corresponds to the transcript of the endogenous gene. Typically, such a nucleotide sequence has substantial sequence identity to the sequence of the transcript of the endogenous gene, preferably greater than about 65% sequence identity, more preferably greater than about 85% sequence identity, most preferably greater than about 95% sequence identity. See U.S. Pat. Nos. 5,283,184 and 5,034,323; herein incorporated by reference.

In some embodiments, the content and/or composition of polypeptides of the present invention in a plant may be modulated by altering, in vivo or in vitro, the promoter of the nucleotide sequence to up- or down-regulate expression. For instance, an isolated nucleic acid comprising a promoter sequence operably linked to a polynucleotide of the present invention is transfected into a plant cell. Subsequently, a plant cell comprising the promoter operably linked to a polynucleotide of the present invention is selected for by means known to those of skill in the art such as, but not limited to, Southern blot, DNA sequencing, or PCR analysis using primers specific to the promoter and to the gene and detecting amplicons produced therefrom. A plant or plant part altered or modified by the foregoing embodiments is grown under plant forming conditions for a time sufficient to modulate the concentration and/or composition of polypeptides of the present invention in the plant. Plant forming conditions are well known in the art and discussed briefly, supra. Detection of expression of a polypeptide of the invention occurs through any method known to one of skill in the art including, but not limited to, immunolocalization.

In general, concentration or composition of the polypeptides of the invention is increased or decreased by at least 5%, 10%, 20%, 30%, 40%, 50%, 60%, 70%, 80%, or 90% relative to a native control plant, plant part, or cell lacking the aforementioned recombinant expression cassette. Modulation in the present invention may occur during and/or subsequent to growth of the plant to the desired stage of development. Modulating nucleic acid expression temporally and/or in particular tissues can be controlled by employing the appropriate promoter operably linked to a polynucleotide of the present invention in, for example, sense or antisense orientation as discussed in greater detail, supra. Induction of expression of a polynucleotide of the present invention can also be controlled by exogenous administration of an effective amount of inducing compound. Inducible promoters and inducing compounds, which activate expression from these promoters, are well known in the art. In various embodiments, the polypeptides of the present invention are modulated in crop plants, particularly maize, wheat, soybean, alfalfa, barley, oats, and rice.

The methods of the invention can be used with other methods available in the art for enhancing disease resistance in plants. Similarly, the antimicrobial compositions described herein may be used alone or in combination with other nucleotide sequences, polypeptides, or agents to protect against plant diseases and pathogens. Although any one of a variety of second nucleotide sequences may be utilized, specific embodiments of the invention encompass those second nucleotide sequences that, when expressed in a plant, help to increase the resistance of a plant to pathogens.

Proteins, peptides, and lysozymes that naturally occur in insects (Jaynes et al. (1987) Bioassays 6:263-270), plants (Broekaert et al. (1997) Critical Reviews in Plant Sciences 16:297-323), animals (Vunnam et al. (1997) J. Peptide Res. 49:59-66), and humans (Mitra and Zang (1994) Plant Physiol. 106:977-981; Nakajima et al. (1997) Plant Cell Reports 16:674-679) are also a potential source of plant disease resistance. Examples of such plant resistance-conferring sequences include those encoding sunflower rhoGTPase-Activating Protein (rhoGAP), lipoxygenase (LOX), Alcohol Dehydrogenase (ADH), and Sclerotinia-Inducible Protein-1 (SCIP-1) described in U.S. application Ser. No. 09/714,767, herein incorporated by reference. These nucleotide sequences enhance plant disease resistance through the modulation of development, developmental pathways, and the plant pathogen defense system. Other plant defense proteins include those described in WO 99/43823 and WO 99/43821, all of which are herein incorporated by reference. It is recognized that such second nucleotide sequences may be used in either the sense or antisense orientation depending on the desired outcome.

In another embodiment, the plant defense proteins comprise isolated polypeptides of the invention. The plant defense proteins of the invention find use in the decontamination of plant pathogens during the processing of grain for animal or human food consumption; during the processing of feedstuffs, and during the processing of plant material for silage. In this embodiment, the plant defense proteins of the invention are presented to grain, plant material for silage, or a contaminated food crop, or during an appropriate stage of the processing procedure, in amounts effective for antimicrobial activity. The compositions can be applied to the environment of a plant pathogen by, for example, spraying, atomizing, dusting, scattering, coating or pouring, introducing into or on the soil, introducing into irrigation water, by seed treatment, or dusting at a time when the plant pathogen has begun to appear or before the appearance of pests as a protective measure. It is recognized that any means that bring the defensive agent polypeptides in contact with the plant pathogen can be used in the practice of the invention.

Additionally, the compositions can be used in formulations used for their antimicrobial activities. Methods are provided for controlling plant pathogens comprising applying a decontaminating amount of a polypeptide or composition of the invention to the environment of the plant pathogen. The polypeptides of the invention can be formulated with an acceptable carrier into a composition(s) that is, for example, a suspension, a solution, an emulsion, a dusting powder, a dispersible granule, a wettable powder, an emulsifiable concentrate, an aerosol, an impregnated granule, an adjuvant, a coatable paste, and also encapsulations in, for example, polymer substances.

Such compositions disclosed above may be obtained by the addition of a surface-active agent, an inert carrier, a preservative, a humectant, a feeding stimulant, an attractant, an encapsulating agent, a binder, an emulsifier, a dye, a UV protectant, a buffer, a flow agent or fertilizers, micronutrient donors or other preparations that influence plant growth. One or more agrochemicals including, but not limited to, herbicides, insecticides, fungicides, bacteriocides, nematocides, molluscicides, acaracides, plant growth regulators, harvest aids, and fertilizers, can be combined with carriers, surfactants, or adjuvants customarily employed in the art of formulation or other components to facilitate product handling and application for particular target mycotoxins. Suitable carriers and adjuvants can be solid or liquid and correspond to the substances ordinarily employed in formulation technology, e.g., natural or regenerated mineral substances, solvents, dispersants, wetting agents, tackifiers, binders, or fertilizers. The active ingredients of the present invention are normally applied in the form of compositions and can be applied to the crop area or plant to be treated, simultaneously or in succession, with other compounds. In some embodiments, methods of applying an active ingredient of the present invention or an agrochemical composition of the present invention (which contains at least one of the proteins of the present invention) are foliar application, seed coating, and soil application.

Suitable surface-active agents include, but are not limited to, anionic compounds such as a carboxylate of, for example, a metal; a carboxylate of a long chain fatty acid; an N-acylsarcosinate; mono or di-esters of phosphoric acid with fatty alcohol ethoxylates or salts of such esters; fatty alcohol sulfates such as sodium dodecyl sulfate, sodium octadecyl sulfate, or sodium cetyl sulfate; ethoxylated fatty alcohol sulfates; ethoxylated alkylphenol sulfates; lignin sulfonates; petroleum sulfonates; alkyl aryl sulfonates such as alkyl-benzene sulfonates or lower alkylnaphtalene sulfonates, e.g., butyl-naphthalene sulfonate; salts of sulfonated naphthalene-formaldehyde condensates; salts of sulfonated phenol-formaldehyde condensates; more complex sulfonates such as the amide sulfonates, e.g., the sulfonated condensation product of oleic acid and N-methyl taurine; or the dialkyl sulfosuccinates, e.g., the sodium sulfonate or dioctyl succinate. Non-ionic agents include condensation products of fatty acid esters, fatty alcohols, fatty acid amides or fatty-alkyl- or alkenyl-substituted phenols with ethylene oxide, fatty esters of polyhydric alcohol ethers, e.g., sorbitan fatty acid esters, condensation products of such esters with ethylene oxide, e.g. polyoxyethylene sorbitar fatty acid esters, block copolymers of ethylene oxide and propylene oxide, acetylenic glycols such as 2, 4, 7, 9-tetraethyl-5-decyn-4,7-diol, or ethoxylated acetylenic glycols. Examples of a cationic surface-active agent include, for instance, an aliphatic mono-, di-, or polyamine such as an acetate, naphthenate, or oleate; or oxygen-containing amine such as an amine oxide of polyoxyethylene alkylamine; an amide-linked amine prepared by the condensation of a carboxylic acid with a di- or polyamine; or a quaternary ammonium salt.

Examples of inert materials include, but are not limited to, inorganic minerals such as kaolin, phyllosilicates, carbonates, sulfates, phosphates, or botanical materials such as cork, powdered corncobs, peanut hulls, rice hulls, and walnut shells.

The compositions of the present invention can be in a suitable form for direct application or as concentrate of primary composition, which requires dilution with a suitable quantity of water or other diluent before application. The decontaminating concentration will vary depending upon the nature of the particular formulation, specifically, whether it is a concentrate or to be used directly.

In a further embodiment, the compositions, as well as the polypeptides of the present invention can be treated prior to formulation to prolong the activity when applied to the environment of a plant pathogen as long as the pretreatment is not deleterious to the activity. Such treatment can be by chemical and/or physical means as long as the treatment does not deleteriously affect the properties of the composition(s). Examples of chemical reagents include, but are not limited to, halogenating agents; aldehydes such as formaldehyde and glutaraldehyde; anti-infectives, such as zephiran chloride; alcohols, such as isopropanol and ethanol; and histological fixatives, such as Bouin's fixative and Helly's fixative (see, for example, Humason (1967) Animal Tissue Techniques (W.H. Freeman and Co.)).

In an embodiment of the invention, the compositions of the invention comprise a microbe having stably integrated the nucleotide sequence of a defensive agent. The resulting microbes can be processed and used as a microbial spray. Any suitable microorganism can be used for this purpose. See, for example, Gaertner et al. (1993) in Advanced Engineered Pesticides, Kim (Ed.). In one embodiment, the nucleotide sequences of the invention are introduced into microorganisms that multiply on plants (epiphytes) to deliver the plant defense proteins to potential target crops. Epiphytes can be, for example, gram-positive or gram-negative bacteria.

It is further recognized that whole, i.e., unlysed, cells of the transformed microorganism can be treated with reagents that prolong the activity of the polypeptide produced in the microorganism when the microorganism is applied to the environment of a target plant. A secretion signal sequence may be used in combination with the gene of interest such that the resulting enzyme is secreted outside the microorganism for presentation to the target plant.

In this manner, a gene encoding a defensive agent of the invention may be introduced via a suitable vector into a microbial host, and said transformed host applied to the environment, plants, or animals. Microorganism hosts that are known to occupy the “phytosphere” (phylloplane, phyllosphere, rhizosphere, and/or rhizoplane) of one or more crops of interest may be selected for transformation. These microorganisms are selected so as to be capable of successfully competing in the particular environment with the wild-type microorganisms, to provide for stable maintenance and expression of the gene expressing the detoxifying polypeptide, and for improved protection of the proteins of the invention from environmental degradation and inactivation.

Such microorganisms include bacteria, algae, and fungi. Illustrative prokaryotes, both Gram-negative and -positive, include Enterobacteriaceae, such as Escherichia, Erwinia, Shigella, Salmonella, and Proteus; Bacillaceae; Rhizobiaceae, such as Rhizobium; Spirillaceae, such as photobacterium, Zymomonas, Serratia, Aeromonas, Vibrio, Desulfovibrio, Spirillum; Lactobacillaceae; Pseudomonadaceae, such as Pseudomonas and Acetobacter; Azotobacteraceae; and Nitrobacteraceae. Among eukaryotes are fungi, such as Phycomycetes and Ascomycetes, which includes yeast, such as Saccharomyces and Schizosaccharomyces; and Basidiomycetes yeast, such as Rhodotorula, Aureobasidium, Sporobolomyces, and the like. Of particular interest are microorganisms, such as bacteria, e.g., Pseudomonas, Erwinia, Serratia, Klebsiella, Xanthomonas, Streptomyces, Rhizobium, Rhodopseudomonas, Methylius, Agrobacterium, Acetobacter, Lactobacillus, Arthrobacter, Azotobacter, Leuconostoc, and Alcaligenes; fungi, particularly yeast, e.g., Saccharomyces, Pichia, Cryptococcus, Kluyveromyces, Sporobolomyces, Rhodotorula, Aureobasidium, and Gliocladium. Of particular interest are such phytosphere bacterial species as Pseudomonas syringae, P. fluorescens; Serratia marcescens, Acetobacter xylinum, Agrobacteria, Rhodopseudomonas spheroides, Xanthomonas campestris, Rhizobium melioti, Alcaligenes entrophus, Clavibacter xyli, and Azotobacter vinlandii; and phytosphere yeast species such as Rhodotorula rubra, R. glutinis, R. marina, R. aurantiaca, Cryptococcus albidus, C. diffluens, C. laurentii, Saccharomyces rosei, S. pretoriensis, S. cerevisiae, Sporobolomyces roseus, S. odorus, Kluyveromyces veronae, and Aureobasidium pullulans.

An isolated polypeptide of the invention can be used as an immunogen to generate antibodies that bind the plant defense peptides using standard techniques for polyclonal and monoclonal antibody preparation. The full-length sequences can be used or, alternatively, the invention provides antigenic peptide fragments of the sequences for use as immunogens. The antigenic peptide of a defensive agent comprises at least 8, preferably 10, 15, 20, or 30 amino acid residues of the amino acid sequence shown in SEQ ID NOs: 4, 5, 9, 10, 14, 15, 19, 20, 24, 25, 29, 30, 34, 35, 39, 40, 44, 45, 49, 50, 53, 54, 58, 59, 63, 64, 68, 69, 73, 74, 78, 79, 83, 84, 88, 89, 93, 94, 98, 99, 103, 104, 108, 109, 113, 114, 118, 119, 120, 121, 122, 123 and 124 and encompasses an epitope of a plant defense protein such that an antibody raised against the peptide forms a specific immune complex with the antimicrobial polypeptides. Epitopes encompassed by the antigenic peptide are regions of plant defense peptides that are located on the surface of the protein, e.g., hydrophilic regions, which are readily ascertainable by those of skill in the art.

Accordingly, another aspect of the invention pertains to polyclonal and monoclonal antibodies that bind a plant defense protein. Polyclonal antibodies can be prepared by immunizing a suitable subject (e.g., rabbit, goat, mouse, or other mammal) with an immunogen. The antibody titer in the immunized subject can be monitored over time by standard techniques, such as with an enzyme linked immunosorbent assay (ELISA) using immobilized antimicrobial polypeptides. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985) in Monoclonal Antibodies and Cancer Therapy, ed. Reisfeld and Sell (Alan R. Liss, Inc., New York, N.Y.), pp. 77-96) or trioma techniques. The technology for producing hybridomas is well known (see generally Coligan et al., eds. (1994) Current Protocols in Immunology (John Wiley & Sons, Inc., New York, N.Y.); Galfre et al. (1977) Nature 266:55052; Kenneth (1980) in Monoclonal Antibodies: A New Dimension In Biological Analyses (Plenum Publishing Corp., NY; and Lerner (1981) Yale J. Biol. Med., 54:387-402).

Alternative to preparing monoclonal antibody-secreting hybridomas, a monoclonal antibody can be identified and isolated by screening a recombinant combinatorial immunoglobulin library (e.g., an antibody phage display library) with a plant defense peptide to thereby isolate immunoglobulin library members that bind the defensive agent. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening an antibody display library can be found in, for example, U.S. Pat. No. 5,223,409; PCT Publication Nos. WO 92/18619; WO 91/17271; WO 92/20791; WO 92/15679; 93/01288; WO 92/01047; 92/09690; and 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734. The antibodies can be used to identify homologs of the plant defense peptides of the invention.

The following examples are offered by way of illustration and not by way of limitation.

### EXPERIMENTAL

### Example 1

### Transformation and Regeneration of Transgenic Plants in Maize

Immature maize embryos from greenhouse donor plants are bombarded with a plasmid containing a plant defense nucleotide sequence of the invention operably linked to a ubiquitin promoter and the selectable marker gene PAT (Wohlleben et al. (1988) Gene 70:25-37), which confers resistance to the herbicide Bialaphos. Alternatively, the selectable marker gene is provided on a separate plasmid. Transformation is performed as follows. Media recipes follow below.

### Preparation of Target Tissue

The ears are husked and surface sterilized in 30% Clorox bleach plus 0.5% Micro detergent for 20 minutes, and rinsed two times with sterile water. The immature embryos are excised and placed embryo axis side down (scutellum side up), 25 embryos per plate, on 560Y medium for 4 hours and then aligned within the 2.5-cm target zone in preparation for bombardment.

### Preparation of DNA

A plasmid vector comprising a plant defense nucleotide sequence of the invention operably linked to a ubiquitin promoter is made. This plasmid DNA plus plasmid DNA containing a PAT selectable marker is precipitated onto 1.1 μm (average diameter) tungsten pellets using a CaCl₂ precipitation procedure as follows:

100 μl prepared tungsten particles in water

10 μl (1 μg) DNA in Tris EDTA buffer (1 μg total DNA)

100 μl 2.5 M CaCl₂

10 μl 0.1 M spermidine

Each reagent is added sequentially to the tungsten particle suspension, while maintained on the multitube vortexer. The final mixture is sonicated briefly and allowed to incubate under constant vortexing for 10 minutes. After the precipitation period, the tubes are centrifuged briefly, liquid removed, washed with 500 ml 100% ethanol, and centrifuged for 30 seconds. Again the liquid is removed, and 105 μl 100% ethanol is added to the final tungsten particle pellet. For particle gun bombardment, the tungsten/DNA particles are briefly sonicated and 10 μl spotted onto the center of each macrocarrier and allowed to dry about 2 minutes before bombardment.

### Particle Gun Treatment

The sample plates are bombarded at level #4 in particle gun #HE34-1 or #HE34-2. All samples receive a single shot at 650 PSI, with a total of ten aliquots taken from each tube of prepared particles/DNA.

### Subsequent Treatment

Following bombardment, the embryos are kept on 560Y medium for 2 days, then transferred to 560R selection medium containing 3 mg/liter Bialaphos, and subcultured every 2 weeks. After approximately 10 weeks of selection, selection-resistant callus clones are transferred to 288J medium to initiate plant regeneration. Following somatic embryo maturation (2-4 weeks), well-developed somatic embryos are transferred to medium for germination and transferred to the lighted culture room. Approximately 7-10 days later, developing plantlets are transferred to 272V hormone-free medium in tubes for 7-10 days until plantlets are well established. Plants are then transferred to inserts in flats (equivalent to 2.5″ pot) containing potting soil and grown for 1 week in a growth chamber, subsequently grown an additional 1-2 weeks in the greenhouse, then transferred to classic 600 pots (1.6 gallon) and grown to maturity. Plants are monitored and scored for altered defense response, plant defense activity, insect resistance, nematode resistance, viral resistance, or fungal resistance.

### Bombardment and Culture Media

Bombardment medium (560Y) comprises 4.0 g/l N6 basal salts (SIGMA C-1416), 1.0 ml/l Eriksson's Vitamin Mix (1000× SIGMA-1511), 0.5 mg/l thiamine HCl, 120.0 g/l sucrose, 1.0 mg/l 2,4-D, and 2.88 g/l L-proline (brought to volume with D-1H₂0 following adjustment to pH 5.8 with KOH); 2.0 g/l Gelrite (added after bringing to volume with D-I H₂0); and 8.5 mg/l silver nitrate (added after sterilizing the medium and cooling to room temperature). Selection medium (560R) comprises 4.0 g/l N6 basal salts (SIGMA C-1416), 1.0 ml/l Eriksson's Vitamin Mix (1000× SIGMA-1511), 0.5 mg/l thiamine HCl, 30.0 g/l sucrose, and 2.0 mg/l 2,4-D (brought to volume with D-1H₂0 following adjustment to pH 5.8 with KOH); 3.0 g/l Gelrite (added after bringing to volume with D-1H₂0); and 0.85 mg/l silver nitrate and 3.0 mg/l Bialaphos (both added after sterilizing the medium and cooling to room temperature).

Plant regeneration medium (288J) comprises 4.3 g/l MS salts (GIBCO 11117-074), 5.0 ml/l MS vitamins stock solution (0.100 g nicotinic acid, 0.02 g/l thiamine HCL, 0.10 g/l pyridoxine HCL, and 0.40 g/l glycine brought to volume with polished D-1H₂0) (Murashige and Skoog (1962) Physiol. Plant. 15:473), 100 mg/l myo-inositol, 0.5 mg/l zeatin, 60 g/l sucrose, and 1.0 ml/l of 0.1 mM abscisic acid (brought to volume with polished D-1H₂0 after adjusting to pH 5.6); 3.0 g/l Gelrite (added after bringing to volume with D-1H₂0); and 1.0 mg/l indoleacetic acid and 3.0 mg/l Bialaphos (added after sterilizing the medium and cooling to 60° C.). Hormone-free medium (272V) comprises 4.3 g/l MS salts (GIBCO 11117-074), 5.0 ml/l MS vitamins stock solution (0.100 g/l nicotinic acid, 0.02 g/l thiamine HCL, 0.10 g/l pyridoxine HCL, and 0.40 g/l glycine brought to volume with polished D-1H₂0), 0.1 g/1 myoinositol, and 40.0 g/l sucrose (brought to volume with polished D-1H₂0 after adjusting pH to 5.6); and 6 g/l bacto-agar (added after bringing to volume with polished D-1H₂0), sterilized and cooled to 60° C.

### Example 2

### Agrobacterium-Mediated Transformation in Maize

For Agrobacterium-mediated transformation of maize with a plant defense nucleotide sequence of the invention operably linked to a ubiquitin promoter, preferably the method of Zhao is employed (U.S. Pat. No. 5,981,840, and PCT patent publication WO98/32326; the contents of which are hereby incorporated by reference). Briefly, immature embryos are isolated from maize and the embryos contacted with a suspension of Agrobacterium, where the bacteria are capable of transferring the DNA construct containing the plant defense nucleotide sequence to at least one cell of at least one of the immature embryos (step 1: the infection step). In this step the immature embryos are preferably immersed in an Agrobacterium suspension for the initiation of inoculation. The embryos are co-cultured for a time with the Agrobacterium (step 2: the co-cultivation step). Preferably the immature embryos are cultured on solid medium following the infection step. Following this co-cultivation period an optional “resting” step is contemplated. In this resting step, the embryos are incubated in the presence of at least one antibiotic known to inhibit the growth of Agrobacterium without the addition of a selective agent for plant transformants (step 3: resting step). Preferably the immature embryos are cultured on solid medium with antibiotic, but without a selecting agent, for elimination of Agrobacterium and for a resting phase for the infected cells. Next, inoculated embryos are cultured on medium containing a selective agent and growing transformed callus is recovered (step 4: the selection step). Preferably, the immature embryos are cultured on solid medium with a selective agent resulting in the selective growth of transformed cells. The callus is then regenerated into plants (step 5: the regeneration step), and preferably calli grown on selective medium are cultured on solid medium to regenerate the plants.

### Example 3

### Soybean Embryo Transformation

Soybean embryos are bombarded with a plasmid containing the plant defense nucleotide sequences operably linked to a ubiquitin promoter as follows. To induce somatic embryos, cotyledons, 3-5 mm in length dissected from surface-sterilized, immature seeds of the soybean cultivar A2872, are cultured in the light or dark at 26° C. on an appropriate agar medium for six to ten weeks. Somatic embryos producing secondary embryos are then excised and placed into a suitable liquid medium. After repeated selection for clusters of somatic embryos that multiplied as early, globular-staged embryos, the suspensions are maintained as described below.

Soybean embryogenic suspension cultures can be maintained in 35 ml liquid media on a rotary shaker, 150 rpm, at 26° C. with florescent lights on a 16:8 hour day/night schedule. Cultures are subcultured every two weeks by inoculating approximately 35 mg of tissue into 35 ml of liquid medium.

Soybean embryogenic suspension cultures may then be transformed by the method of particle gun bombardment (Klein et al. (1987) Nature (London) 327:70-73, U.S. Pat. No. 4,945,050). A Du Pont Biolistic PDS 1000/HE instrument (helium retrofit) can be used for these transformations.

A selectable marker gene that can be used to facilitate soybean transformation is a transgene composed of the 35S promoter from Cauliflower Mosaic Virus (Odell et al. (1985) Nature 313:810-812), the hygromycin phosphotransferase gene from plasmid pJR225 (from E. coli; Gritz et al. (1983) Gene 25:179-188), and the 3′ region of the nopaline synthase gene from the T-DNA of the Ti plasmid of Agrobacterium tumefaciens. The expression cassette comprising the plant defense nucleotide sequence operably linked to the ubiquitin promoter can be isolated as a restriction fragment. This fragment can then be inserted into a unique restriction site of the vector carrying the marker gene.

To 50 μl of a 60 mg/ml 1 μm gold particle suspension is added (in order): 5 μl DNA (1 μg/μl), 20 μl spermidine (0.1 M), and 50 μl CaCl₂ (2.5 M). The particle preparation is then agitated for three minutes, spun in a microfuge for 10 seconds and the supernatant removed. The DNA-coated particles are then washed once in 400 μl 70% ethanol and resuspended in 40 μl of anhydrous ethanol. The DNA/particle suspension can be sonicated three times for one second each. Five microliters of the DNA-coated gold particles are then loaded on each macro carrier disk.

Approximately 300-400 mg of a two-week-old suspension culture is placed in an empty 60×15 mm petri dish and the residual liquid removed from the tissue with a pipette. For each transformation experiment, approximately 5-10 plates of tissue are normally bombarded. Membrane rupture pressure is set at 1100 psi, and the chamber is evacuated to a vacuum of 28 inches mercury. The tissue is placed approximately 3.5 inches away from the retaining screen and bombarded three times. Following bombardment, the tissue can be divided in half and placed back into liquid and cultured as described above.

Five to seven days post bombardment, the liquid media may be exchanged with fresh media, and eleven to twelve days post-bombardment with fresh media containing 50 mg/ml hygromycin. This selective media can be refreshed weekly. Seven to eight weeks post-bombardment, green, transformed tissue may be observed growing from untransformed, necrotic embryogenic clusters. Isolated green tissue is removed and inoculated into individual flasks to generate new, clonally propagated, transformed embryogenic suspension cultures. Each new line may be treated as an independent transformation event. These suspensions can then be subcultured and maintained as clusters of immature embryos or regenerated into whole plants by maturation and germination of individual somatic embryos.

### Example 4

### Sunflower Meristem Tissue Transformation

Sunflower meristem tissues are transformed with an expression cassette containing the plant defense sequence operably linked to a ubiquitin promoter as follows (see also European Patent Number EP 0 486233, herein incorporated by reference, and Malone-Schoneberg et al. (1994) Plant Science 103:199-207). Mature sunflower seed (Helianthus annuus L.) are dehulled using a single wheat-head thresher. Seeds are surface sterilized for 30 minutes in a 20% Clorox bleach solution with the addition of two drops of Tween 20 per 50 ml of solution. The seeds are rinsed twice with sterile distilled water.

Split embryonic axis explants are prepared by a modification of procedures described by Schrammeijer et al. (Schrammeijer et al. (1990) Plant Cell Rep. 9: 55-60). Seeds are imbibed in distilled water for 60 minutes following the surface sterilization procedure. The cotyledons of each seed are then broken off, producing a clean fracture at the plane of the embryonic axis. Following excision of the root tip, the explants are bisected longitudinally between the primordial leaves. The two halves are placed, cut surface up, on GBA medium consisting of Murashige and Skoog mineral elements (Murashige et al. (1962) Physiol. Plant., 15: 473-497), Shepard's vitamin additions (Shepard (1980) in Emergent Techniques for the Genetic Improvement of Crops University of Minnesota Press, St. Paul, Minn.), 40 mg/l adenine sulfate, 30 g/l sucrose, 0.5 mg/16-benzyl-aminopurine (BAP), 0.25 mg/l indole-3-acetic acid (IAA), 0.1 mg/l gibberellic acid (GA3), pH 5.6, and 8 g/l Phytagar.

The explants are subjected to microprojectile bombardment prior to Agrobacterium treatment (Bidney et al. (1992) Plant Mol. Biol. 18: 301-313). Thirty to forty explants are placed in a circle at the center of a 60×20 mm plate for this treatment. Approximately 4.7 mg of 1.8 mm tungsten microprojectiles are resuspended in 25 ml of sterile TE buffer (10 mM Tris HCl, 1 mM EDTA, pH 8.0) and 1.5 ml aliquots are used per bombardment. Each plate is bombarded twice through a 150 mm nytex screen placed 2 cm above the samples in a PDS 1000 particle acceleration device.

Disarmed Agrobacterium tumefaciens strain EHA105 is used in all transformation experiments. A binary plasmid vector comprising the expression cassette that contains the plant defense gene operably linked to the ubiquitin promoter is introduced into Agrobacterium strain EHA105 via freeze-thawing as described by Holsters et al. (1978) Mol. Gen. Genet. 163:181-187. This plasmid further comprises a kanamycin selectable marker gene (i.e., nptII). Bacteria for plant transformation experiments are grown overnight (28° C. and 100 RPM continuous agitation) in liquid YEP medium (10 gm/l yeast extract, 10 gm/l Bactopeptone, and 5 gm/l NaCl, pH 7.0) with the appropriate antibiotics required for bacterial strain and binary plasmid maintenance. The suspension is used when it reaches an OD₆₀₀ of about 0.4 to 0.8. The Agrobacterium cells are pelleted and resuspended at a final OD₆₀₀ of 0.5 in an inoculation medium comprised of 12.5 mM MES pH 5.7, 1 gm/l NH₄Cl, and 0.3 gm/l MgSO₄.

Freshly bombarded explants are placed in an Agrobacterium suspension, mixed, and left undisturbed for 30 minutes. The explants are then transferred to GBA medium and co-cultivated, cut surface down, at 26° C. and 18-hour days. After three days of co-cultivation, the explants are transferred to 374B (GBA medium lacking growth regulators and a reduced sucrose level of 1%) supplemented with 250 mg/l cefotaxime and 50 mg/l kanamycin sulfate. The explants are cultured for two to five weeks on selection and then transferred to fresh 374B medium lacking kanamycin for one to two weeks of continued development. Explants with differentiating, antibiotic-resistant areas of growth that have not produced shoots suitable for excision are transferred to GBA medium containing 250 mg/l cefotaxime for a second 3-day phytohormone treatment. Leaf samples from green, kanamycin-resistant shoots are assayed for the presence of NPTII by ELISA and for the presence of transgene expression by assaying for plant defense activity.

NPTII-positive shoots are grafted to Pioneer® hybrid 6440 in vitro-grown sunflower seedling rootstock. Surface sterilized seeds are germinated in 48-0 medium (half-strength Murashige and Skoog salts, 0.5% sucrose, 0.3% gelrite, pH 5.6) and grown under conditions described for explant culture. The upper portion of the seedling is removed, a 1 cm vertical slice is made in the hypocotyl, and the transformed shoot inserted into the cut. The entire area is wrapped with parafilm to secure the shoot. Grafted plants can be transferred to soil following one week of in vitro culture. Grafts in soil are maintained under high humidity conditions followed by a slow acclimatization to the greenhouse environment. Transformed sectors of T₀ plants (parental generation) maturing in the greenhouse are identified by NPTII ELISA and/or by plant defense activity analysis of leaf extracts while transgenic seeds harvested from NPTII-positive T₀ plants are identified by plant defense activity analysis of small portions of dry seed cotyledon.

An alternative sunflower transformation protocol allows the recovery of transgenic progeny without the use of chemical selection pressure. Seeds are dehulled and surface-sterilized for 20 minutes in a 20% Clorox bleach solution with the addition of two to three drops of Tween 20 per 100 ml of solution, then rinsed three times with distilled water. Sterilized seeds are imbibed in the dark at 26° C. for 20 hours on filter paper moistened with water. The cotyledons and root radical are removed, and the meristem explants are cultured on 374E (GBA medium consisting of MS salts, Shepard vitamins, 40 mg/l adenine sulfate, 3% sucrose, 0.5 mg/16-BAP, 0.25 mg/l IAA, 0.1 mg/l GA, and 0.8% Phytagar at pH 5.6) for 24 hours under the dark. The primary leaves are removed to expose the apical meristem, around 40 explants are placed with the apical dome facing upward in a 2 cm circle in the center of 374M (GBA medium with 1.2% Phytagar), and then cultured on the medium for 24 hours in the dark.

Approximately 18.8 mg of 1.8 μm tungsten particles are resuspended in 150 μl absolute ethanol. After sonication, 8 μl of it are dropped on the center of the surface of macrocarrier. Each plate is bombarded twice with 650 PSI rupture discs in the first shelf at 26 mm of Hg helium gun vacuum.

The plasmid of interest is introduced into Agrobacterium tumefaciens strain EHA105 via freeze thawing as described previously. The pellet of overnight-grown bacteria at 28° C. in a liquid YEP medium (10 g/l yeast extract, 10 g/l Bactopeptone, and 5 g/l NaCl, pH 7.0) in the presence of 50 μg/l kanamycin is resuspended in an inoculation medium (12.5 mM 2-mM 2-(N-morpholino) ethanesulfonic acid, MES, 1 g/l NH₄Cl and 0.3 g/l MgSO₄ at pH 5.7) to reach a final concentration of 4.0 at OD 600. Particle-bombarded explants are transferred to GBA medium (374E), and a droplet of bacteria suspension is placed directly onto the top of the meristem. The explants are co-cultivated on the medium for 4 days, after which the explants are transferred to 374C medium (GBA with 1% sucrose and no BAP, IAA, GA3 and supplemented with 250 μg/ml cefotaxime). The plantlets are cultured on the medium for about two weeks under 16-hour day and 26° C. incubation conditions.

Explants (around 2 cm long) from two weeks of culture in 374C medium are screened for plant defense activity using assays known in the art. After positive (i.e., for plant defense gene expression) explants are identified, those shoots that fail to exhibit plant defense activity are discarded, and every positive explant is subdivided into nodal explants. One nodal explant contains at least one potential node. The nodal segments are cultured on GBA medium for three to four days to promote the formation of auxiliary buds from each node. Then they are transferred to 374C medium and allowed to develop for an additional four weeks. Developing buds are separated and cultured for an additional four weeks on 374C medium. Pooled leaf samples from each newly recovered shoot are screened again by the appropriate plant defense protein activity assay. At this time, the positive shoots recovered from a single node will generally have been enriched in the transgenic sector detected in the initial assay prior to nodal culture.

Recovered shoots positive for plant defense activity expression are grafted to Pioneer hybrid 6440 in vitro-grown sunflower seedling rootstock. The rootstocks are prepared in the following manner. Seeds are dehulled and surface-sterilized for 20 minutes in a 20% Clorox bleach solution with the addition of two to three drops of Tween 20 per 100 ml of solution, and are rinsed three times with distilled water. The sterilized seeds are germinated on the filter moistened with water for three days, then they are transferred into 48 medium (half-strength MS salt, 0.5% sucrose, 0.3% gelrite pH 5.0) and grown at 26° C. under the dark for three days, then incubated at 16-hour-day culture conditions. The upper portion of selected seedling is removed, a vertical slice is made in each hypocotyl, and a transformed shoot is inserted into a V-cut. The cut area is wrapped with parafilm. After one week of culture on the medium, grafted plants are transferred to soil. In the first two weeks, they are maintained under high humidity conditions to acclimatize to a greenhouse environment.

### Example 5

### Assaying Plant Defense Activity

The polypeptides described herein may be produced using any number of methods known to those skilled in the art. Such methods include, but are not limited to, expression in bacteria, eukaryotic cell cultures, in planta, and viral expression systems in suitably infected organisms or cell lines. The instant polypeptides may be expressed either as full-length polypeptides, mature forms, or as fusion proteins by covalent attachment to a variety of enzymes, proteins, or affinity tags. Common fusion protein partners include, but are not limited to, glutathione-S-transferase, thioredoxin, maltose binding protein, hexahistidine polypeptides, and chitin binding protein. The fusion proteins may be engineered with a protease recognition site at the fusion point so that fusion partners can be separated by protease digestion to yield intact mature peptides. Examples of such proteases include, but are not limited to, thrombin, enterokinase, and factor Xa. Indeed, any protease which specifically cleaves the peptide connecting the fusion protein and polypeptide of the invention can be used.

Purification of the polypeptides of the invention may utilize any number of separation technologies known to those skilled in the art of protein purification. Examples of such methods include, but are not limited to, homogenization, filtration, centrifugation, heat denaturation, ammonium sulfate precipitation, desalting, pH precipitation, ion exchange chromatography, hydrophobic interaction chromatography, and affinity chromatography. When the polypeptides of the invention are expressed as fusion proteins, the purification protocol may include the use of an affinity resin specific for the fusion protein partner or for the polypeptide of interest. Additional suitable affinity resins may be synthesized by linking the appropriate ligands to a suitable resin such as Sepharose-4B.

Crude, partially purified, or purified polypeptides of the invention, either alone or as a fusion protein, may be utilized in assays to verify expression levels of functional plant defense peptides in host cells and transgenic plants. Assays may be conducted under well known experimental conditions which permit optimal enzymatic activity. See, for example, assays presented by Thevissen, K et al. (1996) J. Biol. Chem. 271:15018-15025 and WO 00/68405, herein incorporated by reference.

### Example 6

### Bioassay Testing the Pesticidal Activity of Polypeptides Against Southern Corn Rootworm (SCRW) and Western Corn Rootworm (WCRW)

Bio-Sery diet (catalog number F9800B, from: BIOSERV, Entomology Division, One 8^{th} Street, Suite 1, Frenchtown, N.J. 08825) is dispensed in 128-well CD International Bioassay trays (catalog number BIO-BA-128 from CD International, Pitman, N.J. 08071).

Protein samples are applied topically to the diet surface. Enough sample material is supplied to provide for replicate observations per sample. The trays are allowed to dry. Rootworms are dispensed into the wells of the bioassay trays. A lid (catalog number BIO-CV-16, CD International, Pitman, N.J., 08071) is placed on each tray, and the trays are placed in an incubator at 26° C. for 4 to 7 days.

For the evaluation of pesticidal activity against SCRW and WCRW, insects are exposed to a solution comprising either buffer (50 mM carbonate buffer (pH 10)) or a solution of protein sample at selected doses, for example, 50 or 5.0 μg/cm².

The bioassays are then scored by counting “live” versus “dead” larvae. Mortality is calculated as a percentage of dead larvae out of the total number of larvae tested.

### Example 7

### Bioassay Testing Pesticidal Activity of Polypeptides Against the Colorado Potato Beetle (Leptinotarsa decemlineata)

Briefly, bioassay parameters are as follows: Bio-Sery diet (catalog number F9800B, from: BIOSERV, Entomology Division, One 8th Street, Suite 1, Frenchtown, N.J. 08825) is dispensed in a 96 well microtiter plate (catalog number 353918, Becton Dickinson, Franklin Lakes, N.J. 07417-1886) having a surface area of 0.33 cm². Protein samples of the invention are applied topically to the diet surface. Enough sample material is supplied to provide for 8 observations/sample. After the samples dry, 1 Colorado potato beetle neonate is added to each well providing for a total of 8 larvae/sample. A Mylar® lid (Clear Lam Packaging, Inc., 1950 Pratt Blvd., Elk Grove Village, Ill. 60007-5993) is affixed to each tray. Bioassay trays are placed in an incubator at 25° C. The test is scored for mortality on the 7th day following live infesting.

### Example 8

### Bioassay Testing Pesticidal Activity of Polypeptides Against Lepidopterans

Neonate larvae are reared according to standard protocols, such as those published by Czapla and Lang, J. Economic Entomology 83:2480-2485 (1990). Test compounds are either applied topically to the diet or incorporated into the larvae diet (see Czapla and Lang, J. Economic Entomology 83:2480-2485 (1990)). The larvae diet is dispensed to bioassay trays. One larva is applied per well of the bioassay tray. Weight and mortality are recorded 7 days following the start of the test.

### Example 9

### Homopteran Membrane Feeding Bioassay for Screening Proteins

This assay can be used for a variety of homopterans. The assay involves trapping the sample protein between two layers of maximally stretched parafilm which act as a sachet on top of a small vessel containing the insect of choice.

The assay is prepared as follows: 1 cm diameter polystyrene tubing is cut into 15 mm lengths. One end of the tube is then capped with a fine mesh screen. Five insects are then added to the chamber after which the first layer of parafilm is stretched over the remaining open end. 25 μl of sample (polypeptide in a 5% sucrose solution containing McCormick green food coloring) is then placed on top of the stretched parafilm. A second layer of parafilm is then stretched by hand and placed over the sample. The sample is spread between the two layers of parafilm to make a continuous sachet on which the insects feed. The sachet is then covered tightly with saran wrap to prevent evaporation and produce a slightly pressurized sample. The assay tubes are monitored for insect reproduction and death on a 24 hour basis and compared to the 5% sucrose control.

### Example 10

### Testing of Constructs in C. elegans

E. coli cells transformed with constructs containing the plant defense genes of the embodiments are grown in LB medium with a suitable antibiotic overnight at 37° C. 225 at rpm, then the cultures are diluted five fold with fresh LB plus antibiotic and continuously grown at 37° C. at 225 rpm. When the OD₆₀₀ reaches 0.6, IPTG is added to the culture (final IPTG concentration is 1 mM) to induce protein expression. Uninduced cultures are also prepared as controls. Four hours later, the cultures are collected for running SDS-PAGE and setting up a C. elegans assay. For the assay, 5 to 30 μL, of the liquid culture is added into assay wells in 96-well plates. Each assay well contains 120 μL, of liquid with ˜50 L1 staged C. elegans, an appropriate amount of a selective agent such as one or more antibiotics, D3 overnight culture and S-medium. E. coli strain OP50 is used as a control. Forty eight hours later, the assay plates are scored under a microscope by checking worm growth and development, which will show if the peptide has nematicidal activity.

### Example 11

### In Planta Expression of Plant Defense Proteins, C. Elegans Feeding Assays and Phytotoxicity Testing

In order to check if plant defense proteins can be expressed in plants and if they are phytotoxic, N. benthamiana transient assays are performed with the vector constructs containing the plant defense genes of the embodiments.

Three days after infiltration, total protein is extracted from the infiltrated leaves with Tris extraction buffer (100 mM Tris pH8.0, 100 mM NaCl, 1 mM EDTA, 10 mM DTT and 1× protease inhibitors). The protein samples are checked with SDS-PAGE to check the level of expression of the protein.

The same infiltrated extracts are fed to C. elegans to check for nematicidal activity (as described in Example 10, except protein samples are added and E. coli OP50 is used as food for C. elegans).

### Example 12

### Astragalus Hairy Root Testing

Astragalus seeds are soaked in 90% ethanol for 10 minutes and rinsed with sterile water for 3-4 times. Then the seeds are soaked overnight in 3% PPM (Preservative for plant tissue culture media) in ½ MS (Murashige and Skoog basal medium) liquid medium, and plated on ½ MS agar medium. The plates are sealed and incubated in an incubator with 16 hour light at 26° C. to germinate. Agrobacterium rhizogenes strain K599 is transformed with the binary construct containing the plant defense gene of interest. The transformed K599 is grown at 28° C. until OD₆₀₀ reaches 2.0. The culture is centrifuged at 4000 rpm for 10 minutes to obtain a cell pellet, which is then re-suspended in ½ MS liquid medium. Astragalus hypocotyls are cut and soaked in above suspension for 10 minutes and transferred onto sterilized filter paper to soak up excess Agrobacterium cells. The hypocotyls are transferred to ½ MS plates with one piece of filter paper and the plates are sealed and placed into the incubator.

Once hairy roots grow from the hypocotyls, individual roots are transferred (each root represents a line) onto MS-KT (Kanamycin and Timentin) plates, which are put in a dark incubator at 26° C. After about 2 weeks, the healthy roots (transformed) are transferred onto fresh MS-T+0.05% PPM with a single line per plate. After 4 weeks, fresh root tips about 2-4 cm long are cut and transferred to 10 plates to propagate the hairy roots. When the plates are about half full, they are inoculated with about 2000 Meloidogyne incognita eggs. Nematode infection is scored in 6-8 weeks by bleaching the roots and counting egg numbers in each plate.

### Example 13

### Soybean Hairy Root Testing

### Production and Assay of Transformed Soybean Root Cultures

Agrobacterium rhizogenes strain K599 is used for soybean hairy root transformation, and the gene function and promoter activity are analyzed in transgenic soybean hairy roots. Stocks of A. rhizogenes are maintained on minimal A media (see recipes, below). Plasmid DNA is introduced into A. rhizogenes strain K599 using the freeze-thaw method, as described in Ha (1988) Plant Molecular Manual, eds. Gelvin, Schilperoort, and Verma, pp. A3/1-A3/7.

### Generation of Transformed Hairy Roots

The methods of Cho et al. (2000; Planta 210:195-204) are essentially followed for the generation of transformed hairy roots. Soybean seeds are sterilized in a 50% solution of common household bleach with one drop of Tween 20 detergent added. The seeds are then planted on soil that has been autoclaved in containers that can also be used for growing. 15 mL of water are provided. The seeds are allowed to germinate and grow for approximately a week after which cotyledons are harvested.

In order to harvest cotyledons, a sterile forceps and scissors are used to grasp the plant above the cotyledon. The stem is cut just below the node region removing it from the rest of the plant. A second cut is made just above the node region, releasing the cotyledon pair. These are severed by slicing down the center of the stem and placed onto wet filter paper, 6 cotyledons to a 100 mm Petri dish.

A culture of transformed Agrobacterium from the previous example is prepared the day before the cotyledon harvest by inoculating 20 mL of liquid 557A media (10.5 g/L potassium phosphate dibasic, 4.5 g/L potassium phosphate monobasic, 1.0 g/L ammonium sulfate, 0.5 g/L sodium citrate dihydrate, g/L sucrose, 0.1 g/L magnesium sulfate) containing kanamycin as a selective agent at 100 mg/L in a 125 mL flask with Agrobacterium carrying the construct of previous example. The culture is grown overnight at 28° C. On the day of the transformation the optical density is adjusted to 0.3 to 0.5 using 557A liquid media at the same kanamycin concentration. When the culture reaches OD₅₅₀ between 0.6 and 0.7, 5 mL of the culture are placed into a 15 mL rounded centrifuge tube, spun at 4500 rpm (2790×g) for 10 minutes and the supernatant decanted. 5 mL of coculture media 552A (2.5 g/L potassium nitrate, 150 mg/L calcium chloride dihydrate, 250 mg/L magnesium sulfate heptahydrate, 314 mg/L ammonium sulfate, 150 mg/L sodium phosphate monobasic monohydrate, 10 mg/L manganese sulfate monohydrate, 3 mg/L boric acid, 0.75 mg/L potassium iodide, 0.25 mg/L sodium molybdate dihydrate, 0.025 mg/L cupric sulfate pentahydrate, 0.025 mg/L cobalt chloride hexahydrate, 10 mg/L thiamine HCl, 1 mg/L pyridoxine HCl, 1 mg/L nicotinic acid, 100 mg/L myo-inositol, 0.037 mg/L disodium EDTA dihydrate, 27.9 mg/L ferrous sulfate heptahydrate, 2 g/L MES Buffer, 20 g/L sucrose) are dispensed into each 15 mL tube. 5 μL of 100 mM acetosyringone are added. The pellet is resuspended by shaking the tube, and the resulting suspension distributed over eight 15 mm×10 mm culture dishes.

Cotyledons are dipped into the Agrobacterium suspensions using a forceps and ensuring that the cut or wounded area makes contact with the solution and then returned to the dish. The dishes are placed in boxes in a dark culture room at 28° C. for 3 days. They are then transferred to plates containing 121T media (4.3 g/L MS Salts, 10 mg/L thiamine HCl, 1 mg/L pyridoxine HCl, 1 mg/L nicotinic acid, 100 mg/L myo-inositol, 30 g/L sucrose, 250 mg/L cefotaxime, 100 mg/L vancomycin) and placed in clear plastic boxes in a light culture room for 10-14 days at 26° C.

### SCN Bioassay

The methods of Cho et al. (supra) are essentially followed for the SCN bioassay. Hairy roots are clipped from the cotyledon and placed onto solid Gamborg's B5 medium (Sigma, PO Box 188178, St. Louis, Mo. 63160 Cat #G5893) with 100 mg/L vancomycin and 250 mg/L cefotaxime to control Agrobacterium cells. Roots which fluoresce green when viewed under a fluorescence microscope using GFP filters (Zeiss: 420 nm excitation, 510 nm emission) are considered to carry the gene being tested; roots which do not are used as negative controls. Lateral roots are collected from each such root to form replicates for that event. These are cultured, two per plate, for several days before inoculations.

Stage 2 SCN juveniles (“J2”) are prepared essentially as described by Hermsmeier et al., (1998); Mol. Plant-Microbe Interact. 11:1258-1263). Briefly, cysts are collected from soybean roots that had been inoculated with SCN eggs in the greenhouse. These are crushed to release eggs and the latter collected in a #500 sieve. Eggs are separated from silt using a 35% sucrose gradient. They are sterilized with 10% bleach solution, then hatched in 3.14 mM ZnSO₄ for 7 days in a hatching chamber composed of a plastic dish containing the ZnSO₄ solution over which a 635-mesh nylon screen is placed. The eggs are placed on top of the screen to allow contact with the air and solution. The J2 swim down into the solution upon hatching.

For inoculation of the stage 2 juveniles onto soybean roots, the methods of Cho et al. (supra) are essentially followed. The J2 nematodes are collected from the hatching chamber, sterilized with 0.001% HgCl₂ for 3 minutes and then suspended in 1% low-gelling agarose at 5000 J2/mL. The agarose is at a temperature of 26-28° C. Each root is covered with 100 μL of this mixture, or approximately 500 J2 SCN nematodes per root segment. The plates are placed into a 28° C. dark growth chamber for 5 to 8 weeks, after which the cysts are counted.

### Example 14

### SCN Bioassay of Transgenic T0 Events

Soybean Cyst Nematodes (SCN) are used to infest transgenic T0 soybean plants in soil. SCN egg inoculum is acquired by harvesting cysts from plants infested 4-6 weeks earlier. Briefly, the soil is rinsed from the roots and passed through nested 20 mesh and 60 mesh screens. The material retained by the 20 mesh screen is discarded but the material retained by the 60 mesh screen is washed thoroughly and the creamy white cysts are recovered (older brown cysts are ignored). Similarly, the plant's root system is scrubbed against the 20 mesh screen nested over the 60 mesh screen. Cysts are harvested from the debris on the 60 mesh screen. Eggs are released from the cysts by means of a dounce homogenizer in the presence of 0.5% Clorox for 2.5 minutes. Following this treatment the eggs are washed with sterile water from the homogenizer onto the surface of a 200 mesh screen. The eggs are then rinsed in water for an additional 5 minutes. Eggs are transferred to a 50 ml conical tube and counted. The eggs are diluted to 5000 eggs/ml. Plants grown in 15 cm conical tubes are inoculated with about 5000 eggs. Plants are maintained in a 26° C. growth chamber with 12:12 light:dark cycle for 1 month prior to harvest and counting of cysts.

### Example 15

### Bioactivity of Polypeptides Against Fungal Pathogens

Antifungal activity of SEQ ID NO: 64 and 94 against Fusarium graminearum and Colletotrichum graminicola was assessed essentially as described in Broekaert et al (1990) (FEMS Microbiol Lett, 1990. 69: p. 55-60). Spores were isolated from sporulating cultures growing on synthetic nutrient poor agar (F. graminearum) or V8 agar (C. graminicola) by washing with ½ PDB solution. The spore concentration was determined using a hemocytometer and adjusted to 50,000 spores/mL. Spore suspension (80 μL) was added to the wells of sterile 96-well flat-bottomed microtitre plates along with 20 μL of filter-sterilized (0.22 μm syringe filter; Millipore) protein or water to give final protein concentrations of 0-10 μM. The plates were shaken briefly and placed in the dark at 25° C. without shaking for 40 hours. Hyphal growth was estimated by measuring the optical density at 595 nm using a microtiter plate reader (SpectraMax Pro M2; Molecular Devices). Each test was performed in duplicate, and results are presented in Table 1 below.

TABLE 1
IC50(ppm) for SEQ ID NO: 64 and 94

IC₅₀ (ppm)

| Protein | F. graminearum | C. graminicola |
|---|---|---|
| VP94 (SEQ ID NO: 64) | 54 | 47 |
| VP139 (SEQ ID NO: 94) | 16 | nt |

All publications, patents and patent applications mentioned in the specification are indicative of the level of those skilled in the art to which this invention pertains. All publications, patents and patent applications are herein incorporated by reference to the same extent as if each individual publication, patent, or patent application was specifically and individually indicated to be incorporated by reference.

Although the foregoing invention has been described in some detail by way of illustration and example, for purposes of clarity of understanding, it will be obvious that certain changes and modifications may be practiced within the scope of the appended claims.

That which is claimed:

1. An isolated nucleic acid molecule comprising a nucleotide sequence selected from the group consisting of:
(a) a nucleotide sequence set forth in SEQ ID NO: 1, 2, 3, 6, 7, 8, 11, 12, 13, 16, 17, 18, 21, 22, 23, 26, 27, 28, 31, 32, 33, 36, 37, 38, 41, 42, 43, 46, 47, 48, 51, 52, 55, 56, 57, 60, 61, 62, 65, 66, 67, 70, 71, 72, 75, 76, 77, 80, 81, 82, 85, 86, 87, 90, 91, 92, 95, 96, 97, 100, 101, 102, 105, 106, 107, 110, 111, 112, 115, 116, and 117;
(b) a nucleotide sequence that encodes a polypeptide having the amino acid sequence set forth in SEQ ID NO: 4, 5, 9, 10, 14, 15, 19, 20, 24, 25, 29, 30, 34, 35, 39, 40, 44, 45, 49, 50, 53, 54, 58, 59, 63, 64, 68, 69, 73, 74, 78, 79, 83, 84, 88, 89, 93, 94, 98, 99, 103, 104, 108, 109, 113, 114, 118, 119, 120, 121, 122, 123 and 124;
(c) a nucleotide sequence that encodes a mature polypeptide having the amino acid sequence set forth in SEQ ID NO: 5, 10, 15, 20, 25, 30, 35, 40, 45, 50, 54, 59, 64, 69, 74, 79, 34, 89, 94, 99, 104, 109, 114, and 119;
(d) a nucleotide sequence that encodes a polypeptide having at least about 70 percent identity to the amino acid sequence set forth in SEQ ID NO: 4, 5, 9, 10, 14, 15, 19, 20, 24, 25, 29, 30, 34, 35, 39, 40, 44, 45, 49, 50, 53, 54, 58, 59, 63, 64, 68, 69, 73, 74, 78, 79, 83, 84, 88, 89, 93, 94, 98, 99, 103, 104, 108, 109, 113, 114, 118, 119, 120, 121, 122, 123 and 124, wherein said polypeptide retains plant defense activity;
(e) a nucleotide sequence that hybridizes under stringent conditions to a nucleotide sequence having a sequence set forth in SEQ ID NO: 1, 2, 3, 6, 7, 8, 11, 12, 13, 16, 17, 18, 21, 22, 23, 26, 27, 28, 31, 32, 33, 36, 37, 38, 41, 42, 43, 46, 47, 48, 51, 52, 55, 56, 57, 60, 61, 62, 65, 66, 67, 70, 71, 72, 75, 76, 77, 80, 81, 82, 85, 86, 87, 90, 91, 92, 95, 96, 97, 100, 101, 102, 105, 106, 107, 110, 111, 112, 115, 116, and 117, wherein said nucleotide sequence encodes a polypeptide that retains plant defense activity; and
(f) a nucleotide sequence consisting of a complement of any one of the nucleotide sequences in (a), (b), (c), (d), or (e).

2. A DNA construct comprising a nucleotide sequence of claim 1, wherein said nucleotide sequence is operably linked to a promoter that drives expression in a host cell.

3. An expression cassette comprising the DNA construct of claim 2.

4. A host cell having stably incorporated into its genome at least one DNA construct of claim 2, wherein said promoter is a heterologous promoter that drives expression in the host cell.

5. The expression cassette of claim 3 further comprising an operably linked polynucleotide encoding a signal peptide.

6. The host cell of claim 4, wherein said host cell is a plant cell.

7. A plant having stably incorporated into its genome the DNA construct of claim 2.

8. Seed of the plant of claim 7, wherein the seed comprise the construct.

9. The plant of claim 7, wherein said plant displays increased resistance to a plant pathogen.

10. The plant of claim 9, wherein said plant pathogen is a fungus.

11. The plant of claim 7, wherein said promoter is a tissue-preferred promoter.

12. The plant of claim 11, wherein said tissue-preferred promoter is selected from the group consisting of a leaf-preferred promoter, a root-preferred promoter, a seed-preferred promoter, a stalk-preferred promoter, and a vascular tissue-preferred promoter.

13. The plant of claim 7, wherein said promoter is a pathogen-inducible promoter.

14. A method for inducing plant pathogen resistance in a plant, said method comprising introducing into a plant at least one expression cassette of claim 3.


SEQUENCE LISTING (124 sequences)

SEQ ID NO: 1 - DNA, 601 bp, Triticum aestivum
  misc_feature (0)...(0): VP-41 TA-PDF32_entire NT seq
gcacgaggtacaatcccagataagtgtacattttacagggtgcgttttagcagatacaaataaggaagaatggagtcatcacacaagcttttcccggccgtagccatcctcctcctgctcgtcgtcgccaccgaggtggtgccagcgcaggcacgagagtgtgagacagagagcgagcggttcaacgggctgtgcttcgtgtccgcaaactgcgccggtgtgtgcaatgcggaggggttcaccggtggcaagtgctccggcttgaagaggagctgcatgtgcacgaaggagtgctagacgatatcatgatatatttaggtggttggatggcgacaattatgaacttattcttcgcatggtttgaattacttttgttcgtatgtctgataaagttccaggttcaccgcgacgttatccttgggttcgaatgaaggaaaatgttttctttctttaaagcaaagaaaatgtgtggcccatgagagtgttcatggaatcacatgtatctccttttttccctgtatttgtctcttctttccgggtgtgcctagctcctatctaaatgagacgtaactaagtatcaaaaaaaaaaaaaaaaaaaaaa

SEQ ID NO: 2 - DNA, 228 bp, Triticum aestivum
  misc_feature (0)...(0): VP-41 TA-PDF32_coding region for complete protein
atggagtcatcacacaagcttttcccggccgtagccatcctcctcctgctcgtcgtcgccaccgaggtggtgccagcgcaggcacgagagtgtgagacagagagcgagcggttcaacgggctgtgcttcgtgtccgcaaactgcgccggtgtgtgcaatgcggaggggttcaccggtggcaagtgctccggcttgaagaggagctgcatgtgcacgaaggagtgctag

SEQ ID NO: 3 - DNA, 141 bp, Triticum aestivum
  misc_feature (0)...(0): VP-41 TA-PDF32_coding region for mature protein
cgagagtgtgagacagagagcgagcggttcaacgggctgtgcttcgtgtccgcaaactgcgccggtgtgtgcaatgcggaggggttcaccggtggcaagtgctccggcttgaagaggagctgcatgtgcacgaaggagtgc

SEQ ID NO: 4 - protein, 75 aa, Triticum aestivum
  PEPTIDE (0)...(0): VP-41 TA-PDF32_complete predicted protein
MESSHKLFPAVAILLLLVVATEVVPAQARECETESERFNGLCFVSANCAGVCNAEGFTGGKCSGLKRSCMCTKEC

SEQ ID NO: 5 - protein, 47 aa, Triticum aestivum
  PEPTIDE (0)...(0): VP-41 TA-PDF32_predicted mature protein
RECETESERFNGLCFVSANCAGVCNAEGFTGGKCSGLKRSCMCTKEC

SEQ ID NO: 6 - DNA, 529 bp, Vernonia mespilifolia
  misc_feature (0)...(0): VP-43 VM-PDF1_entire NT seq
cttgagcttcattctaattcaaaaatggtgcaaaaatcgattgttttctccgcgttccttctaatcctctttatctcagaaatctcgagtgtgagagcagagctatgcgagaaagctagcaagacatggtcaggcaactgtggcaacacaggacattgtgataatcagtgtaagtcatgggagggtgcagcccatggagcttgtcatgtgcgtggagggaaacacatgtgcttttgttatttcaattgtaaaaaagctgaaaaactcgctcaagataagctaaaagcagaagagcttgctaaagacaaactcaaggcagataagtttgaccatgatgcaaaagaagtagtaccaaatgtcgaacatccatgaaagatcggtttccttaaatcaatagctgttttaataagttatgaataaaaacagaaagtgttgtataatcatatttttagcttccttagagatgcattatgttgcaantccacaacttcttgtggtaaatgtgtaaaatgtangatacnaaagctan

SEQ ID NO: 7 - DNA, 348 bp, Vernonia mespilifolia
  misc_feature (0)...(0): VP-43 VM-PDF1_coding region for complete protein
atggtgcaaaaatcgattgttttctccgcgttccttctaatcctctttatctcagaaatctcgagtgtgagagcagagctatgcgagaaagctagcaagacatggtcaggcaactgtggcaacacaggacattgtgataatcagtgtaagtcatgggagggtgcagcccatggagcttgtcatgtgcgtggagggaaacacatgtgcttttgttatttcaattgtaaaaaagctgaaaaactcgctcaagataagctaaaagcagaagagcttgctaaagacaaactcaaggcagataagtttgaccatgatgcaaaagaagtagtaccaaatgtcgaacatccatga

SEQ ID NO: 8 - DNA, 150 bp, Vernonia mespilifolia
  misc_feature (0)...(0): VP-43 VM-PDF1_coding region for mature protein
gagctatgcgagaaagctagcaagacatggtcaggcaactgtggcaacacaggacattgtgataatcagtgtaagtcatgggagggtgcagcccatggagcttgtcatgtgcgtggagggaaacacatgtgcttttgttatttcaattgt

SEQ ID NO: 9 - protein, 115 aa, Vernonia mespilifolia
  PEPTIDE (0)...(0): VP-43 VM-PDF1_complete predicted protein
MVQKSIVFSAFLLILFISEISSVRAELCEKASKTWSGNCGNTGHCDNQCKSWEGAAHGACHVRGGKHMCFCYFNCKKAEKLAQDKLKAEELAKDKLKADKFDHDAKEVVPNVEHP

SEQ ID NO: 10 - protein, 50 aa, Vernonia mespilifolia
  PEPTIDE (0)...(0): VP-43 VM-PDF1_predicted mature protein
ELCEKASKTWSGNCGNTGHCDNQCKSWEGAAHGACHVRGGKHMCFCYFNC

SEQ ID NO: 11 - DNA, 652 bp, Zea mays
  misc_feature (0)...(0): VP-52 ZM-BBPI-1_entire NT seq
caacgaagctagctcttgcttctacgagaggatatagagaggaagaacagctcgtctgacgaccagctggtcgatcgccgccggccgagaccatgaagagcagcccacacctggtgctgatcctgtgcctccaggccgctctggtcatgggcgtcttcgccgctttggctaaagaaaatgccatggtcgagagcaaggccatcgacatcaacccggggcagctcaagtgctgcaccaactgcaacttctccttctcggggctctacacctgcgacgacgtcaaaaaggactgcgaccccgtctgcaagaagtgcgtcgtcgccgtgcacgcctcctactcgggcaacaacaagttcaggtgcaccgacaccttcctcggcatgtgcggccccaagtgctagatcagagaggaagaacgcgcgctgctgctagctgctattagcttcgttcttgcgccggccggcccggtgcatggttcacgtactgtgtgttgtgctactacgatacgtactggcttcttgtgtgttgggttcttgttcttctctcgacagtgcaccttgcgcgccaacaataaaccgttgtttgccatgaataaatagtactatgaagttatttgcccccaaaaaaaaaaaaaaaaaaaaaaaaaaaaaaa

SEQ ID NO: 12 - DNA, 306 bp, Zea mays
  misc_feature (0)...(0): VP-52 ZM-BBPI-1_coding region for complete protein
atgaagagcagcccacacctggtgctgatcctgtgcctccaggccgctctggtcatgggcgtcttcgccgctttggctaaagaaaatgccatggcgagagcaaggccatcgacatcaacccggggcagctcaagtgctgcaccaactgcaacttctccttctcggggctctacacctgcgacgacgtaaaaaggactgcgaccccgtctgcaagaagtgcgtcgtcgccgtgcacgcctcctactcgggcaacaacaagttcaggtgcaccgacaccttctcggcatgtgcggccccaagtgctag

SEQ ID NO: 13 - DNA, 237 bp, Zea mays
  misc_feature (0)...(0): VP-52 ZM-BBPI-1_coding region for mature protein
gctttggctaaagaaaatgccatggcgagagcaaggccatcgacatcaacccggggcagctcaagtgctgcaccaactgcaacttctccttctcggggctctacacctgcgacgacgtaaaaaggactgcgaccccgtctgcaagaagtgcgtcgtcgccgtgcacgcctcctactcgggcaacaacaagttcaggtgcaccgacaccttctcggcatgtgcggccccaagtgctag

SEQ ID NO: 14 - protein, 102 aa, Zea mays
  PEPTIDE (0)...(0): VP-52 ZM-BBPI-1_complete predicted protein
MKSSPHLVLILCLQAALVMGVFAALAKENAMVESKAIDINPGQLKCCTNCNFSFSGLYTCDDVKKDCDPVCKKCVVAVHASYSGNNKFRCTDTFLGMCGPKC

SEQ ID NO: 15 - protein, 77 aa, Zea mays
  PEPTIDE (0)...(0): VP-52 ZM-BBPI-1_predicted mature protein
ALAKENAMARARPSTSTRGSSSAAPTATSPSRGSTPATTKGLRPRLQEVRRRRARLLLGQQQVQVHRHLLGMCGPKC

SEQ ID NO: 16 - DNA, 672 bp, Zea mays
  misc_feature (0)...(0): VP-53 ZM-BBPI-4_entire NT seq
tctcagcagcactagcagtgacaaactcgaagcttgccttcttgtttacaccctaaaactaaaactagagtcgtcggcggcaacaagcggtacgtacgtagaggatgaagagcaggagcagcactctgttggtgatcctagttctccaggcccttctgctctctgcggctgtggccaaaggacctgcagggccgaagaagcagtgctggtgcggcgagtgcaccagctggtcgggcgtgtggacctgcgacgacctcctcaccaagtgcgccgccacctgcaagaactgcgtccccgtgtccacgggcaagggcgccaccaagtacaggtgccgcgacttcctccccgaaaactgcgggtgcaagatccactaaactcatccaattccaccatggccacagcagccgatggatccttcttccatatgcatgttctcctccgtccgtccgcctagctttgctacaagcaggcgctaataagcaagctgctgttgttgttttgcatgtttgattaggtgaccctttccactcgagatggaataaaataaacgttaggcgcacgtactgacgtgttattacgatatttctcccctaagataaaggctctgttagattcgcttcgctgactaacttaccatattttattttaaaaaaaaaaaaaaaaaaaaaaaaa

SEQ ID NO: 17 - DNA, 270 bp, Zea mays
  misc_feature (0)...(0): VP-53 ZM-BBPI-4_coding region for complete protein
atgaagagcaggagcagcactctgttggtgatcctagttctccaggcccttctgctctctgcggctgtggccaaaggacctgcagggccgaagaagcagtgctggtgcggcgagtgcaccagctggtcgggcgtgtggacctgcgacgacctcctcaccaagtgcgccgccacctgcaagaactgcgtccccgtgtccacgggcaagggcgccaccaagtacaggtgccgcgacttcctccccgaaaactgcgggtgcaagatccactaa

SEQ ID NO: 18 - DNA, 198 bp, Zea mays
  misc_feature (0)...(0): VP-53 ZM-BBPI-4_coding region for mature protein
aaaggacctgcagggccgaagaagcagtgctggtgcggcgagtgcaccagctggtcgggcgtgtggacctgcgacgacctcctcaccaagtgcgccgccacctgcaagaactgcgtccccgtgtccacgggcaagggcgccaccaagtacaggtgccgcgacttcctccccgaaaactgcgggtgcaagatccactaa

SEQ ID NO: 19 - protein, 122 aa, Zea mays
  PEPTIDE (0)...(0): VP-53 ZM-BBPI-4_complete predicted protein
MKSRSSTLLVILVLQALLLSAAVAKGPAGPKKQCWCGECTSWSGVWTCDDLLTKCAATCKNCVPVSTGKGATKYRCRDFLPENCGCKIHTHPIPPWPQQPMDPSSICMFSSVRPPSFATSRR

SEQ ID NO: 20 - protein, 65 aa, Zea mays
  PEPTIDE (0)...(0): VP-53 ZM-BBPI-4_predicted mature protein
KGPAGPKKQCWCGECTSWSGVWTCDDLLTKCAATCKNCVPVSTGKGATKYRCRDFLPENCGCKIH

SEQ ID NO: 21 - DNA, 549 bp, Zea mays
  misc_feature (0)...(0): VP-54 ZM-BBPI-6_entire NT seq
gtgacgaactcgaagcttgcgttcttgtagtagaggtcggcagcaagcggtggtagagcgagaggtcgagaagatgaagagcagcactctgttggcgatcctagttctccaggcccttctggtctctgcggccgtggccaaaggacctgcagggccgacgacgaagaagcagtgctggtgcggcgagtgcaccagctggtcgggcgtgtggacctgcgacgacctcctcaccaagtgcgccgccacctgcaagaactgcgtccccgtgtccacggacaagggggccatcaagtacaggtgccgcgacttcctccccgaaaactgcggctgcaagatccactagagactcatccaattccaccatggccgcgcgccacagcggcacagccgatggatccttccatgttcctccgtccgtccgccttgctacaagcaggcagccacaccaataagctagctcttgttgttgttttgtatgtttgattgggtgtcccttcccagtcgagatggaataaaataaatcatcaggcgccaaaaaaaaaaaaaaaa

SEQ ID NO: 22 - DNA, 270 bp, Zea mays
  misc_feature (0)...(0): VP-54 ZM-BBPI-6_coding region for complete protein
atgaagagcagcactctgttggcgatcctagttctccaggcccttctggtctctgcggccgtggccaaaggacctgcagggccgacgacgaagaagcagtgctggtgcggcgagtgcaccagctggtcgggcgtgtggacctgcgacgacctcctcaccaagtgcgccgccacctgcaagaactgcgtccccgtgtccacggacaagggggccatcaagtacaggtgccgcgacttcctccccgaaaactgcggctgcaagatccactag

SEQ ID NO: 23 - DNA, 204 bp, Zea mays
  misc_feature (0)...(0): VP-54 ZM-BBPI-6_coding region for mature protein
aaaggacctgcagggccgacgacgaagaagcagtgctggtgcggcgagtgcaccagctggtcgggcgtgtggacctgcgacgacctcctcaccaagtgcgccgccacctgcaagaactgcgtccccgtgtccacggacaagggggccatcaagtacaggtgccgcgacttcctccccgaaaactgcggctgcaagatccactag

SEQ ID NO: 24 - protein, 89 aa, Zea mays
  PEPTIDE (0)...(0): VP-54 ZM-BBPI-6_complete predicted protein
MKSSTLLAILVLQALLVSAAVAKGPAGPTTKKQCWCGECTSWSGVWTCDDLLTKCAATCKNCVPVSTDKGAIKYRCRDFLPENCGCKIH

SEQ ID NO: 25 - protein, 67 aa, Zea mays
  PEPTIDE (0)...(0): VP-54 ZM-BBPI-6_predicted mature protein
KGPAGPTTKKQCWCGECTSWSGVWTCDDLLTKCAATCKNCVPVSTDKGAIKYRCRDFLPENCGCKIH

SEQ ID NO: 26 - DNA, 551 bp, Zea mays
  misc_feature (0)...(0): VP-56 ZM-BBPI-9_entire NT seq
ctggcctgttcttaattattgccaggacaggagaaacaaacaaagatgaggcctcagctgatactcgtcggcactctggctgttctcgccatcctcgcagctctcggcgaaggctcgtcgtcctggccgtgctgcaacaactgcggtgcttgcaacaggaagcagccgcctgagtgccagtgcaatgacgtgtcggtgaacgggtgccatccggagtgcatgaactgcgtcaaggtcggtgcaggaattcgtcccggcatgggccccggccccgtcgtcacctaccgctgtgatgacgttctcacaaacttctgccagagcagctgcccggaggcgtagttgctgggtggtggtgtcttcttctgacgccatgggacgccagtacgcaaccagtttgcttctctccagcttcgtcagacaagaaatagataaataaacaaatgtcaccggccgctctgttcggtgcttgctcttgttcgtcgtcagagaagaaatagataaataaataaataaataaataaatagccaaaaaaaaaaaaaaaaaaaaaaaa

SEQ ID NO: 27 - DNA, 294 bp, Zea mays
  misc_feature (0)...(0): VP-56 ZM-BBPI-9_coding region for complete protein
atgaggcctcagctgatactcgtcggcactctggctgttctcgccatcctcgcagctctcggcgaaggctcgtcgtcctggccgtgctgcaacaactgcggtgcttgcaacaggaagcagccgcctgagtgccagtgcaatgacgtgtcggtgaacgggtgccatccggagtgcatgaactgcgtcaaggtcggtgcaggaattcgtcccggcatgggccccggccccgtcgtcacctaccgctgtgatgacgttctcacaaacttctgccagagcagctgcccggaggcgtag

SEQ ID NO: 28 - DNA, 216 bp, Zea mays
  misc_feature (0)...(0): VP-56 ZM-BBPI-9_coding region for mature protein
tggccgtgctgcaacaactgcggtgcttgcaacaggaagcagccgcctgagtgccagtgcaatgacgtgtcggtgaacgggtgccatccggagtgcatgaactgcgtcaaggtcggtgcaggaattcgtcccggcatgggccccggccccgtcgtcacctaccgctgtgatgacgttctcacaaacttctgccagagcagctgcccggaggcgtag

SEQ ID NO: 29 - protein, 97 aa, Zea mays
  PEPTIDE (0)...(0): VP-56 ZM-BBPI-9_complete predicted protein
MRPQLILVGTLAVLAILAALGEGSSSWPCCNNCGACNRKQPPECQCNDVSVNGCHPECMNCVKVGAGIRPGMGPGPVVTYRCDDVLTNFCQSSCPEA

SEQ ID NO: 30 - protein, 71 aa, Zea mays
  PEPTIDE (0)...(0): VP-56 ZM-BBPI-9_predicted mature protein
WPCCNNCGACNRKQPPECQCNDVSVNGCHPECMNCVKVGAGIRPGMGPGPVVTYRCDDVLTNFCQSSCPEA

SEQ ID NO: 31 - DNA, 425 bp, Zea mays
  misc_feature (0)...(0): VP-63 ZM-ISC7_entire NT seq
caggtgatctcaagtaattaacagaaaatatattccatcattgaaaatgagttccgttgttttgggtgctactggtagggagaataagacatcatggcctgaggtggtgggcatgtccatcaaggaggcaagagagatcattcttaaagacatgcccaacgctaacattcaagttctaccggttggctcgcttgtgacccaagactttcgccctgatcgtgttcgcatcttcgttgatattgttgcccagactccaacagttggctgacaaggatatgccttatctataggccaaataaacaaagcctacttttatgtatcatggctaataaatcctacatttcttggttaaaaaaaaaaaaaaaaaaaaaaaaaaaaaaaaaaaaaaaaaaaaaaaaaaaaaaaaaaaaaaaaaaaaaaaaaaa

SEQ ID NO: 32 - DNA, 222 bp, Zea mays
  misc_feature (0)...(0): VP-63 ZM-ISC7_coding region for complete protein
atgagttccgttgttttgggtgctactggtagggagaataagacatcatggcctgaggtggtgggcatgtccatcaaggaggcaagagagatcattcttaaagacatgcccaacgctaacattcaagttctaccggttggctcgcttgtgacccaagactttcgccctgatcgtgttcgcatcttcgttgatattgttgcccagactccaacagttggctga

SEQ ID NO: 33 - DNA, 222 bp, Zea mays; identical to SEQ ID NO: 32
  misc_feature (0)...(0): VP-63 ZM-ISC7_Coding region for mature protein

SEQ ID NO: 34 - protein, 73 aa, Zea mays
  PEPTIDE (0)...(0): VP-63 ZM-ISC7_complete predicted protein
MSSVVLGATGRENKTSWPEVVGMSIKEAREIILKDMPNANIQVLPVGSLVTQDFRPDRVRIFVDIVAQTPTVG

SEQ ID NO: 35 - protein, 73 aa, Zea mays; identical to SEQ ID NO: 34
  PEPTIDE (0)...(0): VP-63 ZM-ISC7_predicted mature protein

SEQ ID NO: 36 - DNA, 355 bp, Zea mays
  misc_feature (0)...(0): VP-64 ZM-ISC8_entire NT seq
tagctatccattggacgcagtccgtgagtgaaaatgagccatagccatagcactaagacatcgtggccggaggtggaggggatgcctgctgaggtggcgaagcgcaagatccaggaggaccgcccggacatccaggtgatccttgtgcctgtcgactccgccgtgaccgatgacttcaacaccaagcgcgtccgcgtcttcttcgacaaggccggcaacgtggcacaagtccccaagatcggctagggcaaatagtgtcgttacttcagttctatccagtcgaagctcatcttagttgtatgcggtgttatatacagtattatgcactcgtattttaaaaaaaaaaaaaaaaaaa

SEQ ID NO: 37 - DNA, 213 bp, Zea mays
  misc_feature (0)...(0): VP-64 ZM-ISC8_coding region for complete protein
atgagccatagccatagcactaagacatcgtggccggaggtggaggggatgcctgctgaggtggcgaagcgcaagatccaggaggaccgcccggacatccaggtgatccttgtgcctgtcgactccgccgtgaccgatgacttcaacaccaagcgcgtccgcgtcttcttcgacaaggccggcaacgtggcacaagtccccaagatcggctag

SEQ ID NO: 38 - DNA, 213 bp, Zea mays; identical to SEQ ID NO: 37
  misc_feature (0)...(0): VP-64 ZM-ISC8_coding region for mature protein

SEQ ID NO: 39 - protein, 70 aa, Zea mays
  PEPTIDE (0)...(0): VP-64 ZM-ISC8_complete predicted protein
MSHSHSTKTSWPEVEGMPAEVAKRKIQEDRPDIQVILVPVDSAVTDDFNTKRVRVFFDKAGNVAQVPKIG

SEQ ID NO: 40 - protein, 70 aa, Zea mays; identical to SEQ ID NO: 39
  PEPTIDE (0)...(0): ZM-ISC8_predicted mature protein

SEQ ID NO: 41 - DNA, 826 bp, Zea mays
  misc_feature (0)...(0): VP-65 ZM-IT1B_entire NT seq
caagccaacaactaaaggcaggcgcggcacaccgcgacaccgtaccggcctagacacttggagaaaggagaagcagcgcagcggcatgggcaccagtcccgtcatccctgcgacgatgctcgtcgtcgctctccttgtcgctagtagtactgtctgcttcggcgccaccgccaccgacaccgacaccgacggcgccattcgtctccccagcagctctggtgctgttggtcgaccgtgggagtgctgtgactatgtcaccaaggagccgtttatcaggccgccccggtggcgctgcaacgacgtggtggacaagtgctccgccgactgccaagagtgcgaggagtcgccggccggcgacggcttcgtctgccgtgactggatcttcagcctgctcgagcccccggtctgcacacccaggccgtgggactgctgcgacttcgccgtctgcacaagggactacatcccctactgccagtgcggcgacgtggtggagtcgtgccccagcaactgcaaagcgtgcaagcttgtggagtcggaccctcctcgctaccgatgcctagacgtcttccacggctatcctggtcccaagtgcacgccatggatcagttagagcaattagctcagctcagctacttgctaaataaataaataaaagtagcgcagctcagctacttgctagtacgtagtacgtccacaataaataaataaaagcggctagcgaacaaagctaacacctgtttgttcgtcccgagtcccgacactctttcgcgcaatcgcgcgtcattaaatataaataataactagctaaaaaaaaaaaaaaaaaaaa

SEQ ID NO: 42 - DNA, 525 bp, Zea mays
  misc_feature (0)...(0): VP-65 ZM-IT1B_coding region for complete protein
atgggcaccagtcccgtcatccctgcgacgatgctcgtcgtcgctctccttgtcgctagtagtactgtctgcttcggcgccaccgccaccgacaccgacaccgacggcgccattcgtctccccagcagctctggtgctgttggtcgaccgtgggagtgctgtgactatgtcaccaaggagccgtttatcaggccgccccggtggcgctgcaacgacgtggtggacaagtgctccgccgactgccaagagtgcgaggagtcgccggccggcgacggcttcgtctgccgtgactggatcttcagcctgctcgagcccccggtctgcacacccaggccgtgggactgctgcgacttcgccgtctgcacaagggactacatcccctactgccagtgcggcgacgtggtggagtcgtgccccagcaactgcaaagcgtgcaagcttgtggagtcggaccctcctcgctaccgatgcctagacgtcttccacggctatcctggtcccaagtgcacgccatggatcagttag

SEQ ID NO: 43 - DNA, 447 bp, Zea mays
  misc_feature (0)...(0): VP-65 ZM-IT1B_coding region for mature protein
gccaccgccaccgacaccgacaccgacggcgccattcgtctccccagcagctctggtgctgttggtcgaccgtgggagtgctgtgactatgtcaccaaggagccgtttatcaggccgccccggtggcgctgcaacgacgtggtggacaagtgctccgccgactgccaagagtgcgaggagtcgccggccggcgacggcttcgtctgccgtgactggatcttcagcctgctcgagcccccggtctgcacacccaggccgtgggactgctgcgacttcgccgtctgcacaagggactacatcccctactgccagtgcggcgacgtggtggagtcgtgccccagcaactgcaaagcgtgcaagcttgtggagtcggaccctcctcgctaccgatgcctagacgtcttccacggctatcctggtcccaagtgcacgccatggatcagttag

SEQ ID NO: 44 - protein, 174 aa, Zea mays
  PEPTIDE (0)...(0): VP-65 ZM-IT1B_complete predicted protein
MGTSPVIPATMLVVALLVASSTVCFGATATDTDTDGAIRLPSSSGAVGRPWECCDYVTKEPFIRPPRWRCNDVVDKCSADCQECEESPAGDGFVCRDWIFSLLEPPVCTPRPWDCCDFAVCTRDYIPYCQCGDVVESCPSNCKACKLVESDPPRYRCLDVFHGYPGPKCTPWIS

SEQ ID NO: 45 - protein, 148 aa, Zea mays
  PEPTIDE (0)...(0): VP-65 ZM-IT1B_predicted mature protein
ATATDTDTDGAIRLPSSSGAVGRPWECCDYVTKEPFIRPPRWRCNDVVDKCSADCQECEESPAGDGFVCRDWIFSLLEPPVCTPRPWDCCDFAVCTRDYIPYCQCGDVVESCPSNCKACKLVESDPPRYRCLDVFHGYPGPKCTPWIS

SEQ ID NO: 46 - DNA, 787 bp, Zea mays
  misc_feature (0)...(0): VP-67 ZM-IT3_entire NT seq
ctatactactatcacagtaggaagctaggaggaaatcaaagcaacaaagttgctggccgagagaagcaaccatgagacctcaggcgtcgttactcgtcacactggctgttatcgtcgtcgtccttgcagctctgccactcagcaaagggacggaggaggaaggaggaggaggggaggcagtcgccgccgtggacgccgccgccggaacgagctcgtggccatgctgcaacaagtgcggtttctgctacctgtctgacccgccgcagtgccaatccctggacttctcgacggtcgggtgccacccagagtgcaagcagtgcatcaggtacaccgccgacggtggcgtcgacatcccgcccgtgcacgcctaccgctgcgccgacatcctcttcaacttctgcgagcgccgctgcactcccgccgcagttgctgctagcaccaagtagacggatgcatatgcatgccatcgttgccgccgtgtgtgccgcttcagagaagaactaaataaatgttgccgcgcgctgttgatgcgcgtgcatgcctctccttgaataaaccaatatttctataattattgtaggatgtccaactttcttaagtttgaaaaataaatggcgctgcttcatatgatgctggtgtccatgaattaacctgcatcgtcacttcagttatgatctgtgctttgcatgaggttttcatgttacttttatgtccaagagaacttggattttcttctcgaacggtaaaaaaaaaaaaaaaaaaaaaaaaaaaaaaaaaaaaaaaaaaa

SEQ ID NO: 47 - DNA, 375 bp, Zea mays
  misc_feature (0)...(0): VP-67 ZM-IT3_coding region for complete protein
atgagacctcaggcgtcgttactcgtcacactggctgttatcgtcgtcgtccttgcagctctgccactcagcaaagggacggaggaggaaggaggaggaggggaggcagtcgccgccgtggacgccgccgccggaacgagctcgtggccatgctgcaacaagtgcggtttctgctacctgtctgacccgccgcagtgccaatccctggacttctcgacggtcgggtgccacccagagtgcaagcagtgcatcaggtacaccgccgacggtggcgtcgacatcccgcccgtgcacgcctaccgctgcgccgacatcctcttcaacttctgcgagcgccgctgcactcccgccgcagttgctgctagcaccaagtag

SEQ ID NO: 48 - DNA, 297 bp, Zea mays
  misc_feature (0)...(0): VP-67 ZM-IT3_coding region for mature protein
acggaggaggaaggaggaggaggggaggcagtcgccgccgtggacgccgccgccggaacgagctcgtggccatgctgcaacaagtgcggtttctgctacctgtctgacccgccgcagtgccaatccctggacttctcgacggtcgggtgccacccagagtgcaagcagtgcatcaggtacaccgccgacggtggcgtcgacatcccgcccgtgcacgcctaccgctgcgccgacatcctcttcaacttctgcgagcgccgctgcactcccgccgcagttgctgctagcaccaagtag

SEQ ID NO: 49 - protein, 124 aa, Zea mays
  PEPTIDE (0)...(0): VP-67 ZM-IT3_complete predicted protein
MRPQASLLVTLAVIVVVLAALPLSKGTEEEGGGGEAVAAVDAAAGTSSWPCCNKCGFCYLSDPPQCQSLDFSTVGCHPECKQCIRYTADGGVDIPPVHAYRCADILFNFCERRCTPAAVAASTK

SEQ ID NO: 50 - protein, 98 aa, Zea mays
  PEPTIDE (0)...(0): VP-67 ZM-IT3_predicted mature protein
TEEEGGGGEAVAAVDAAAGTSSWPCCNKCGFCYLSDPPQCQSLDFSTVGCHPECKQCIRYTADGGVDIPPVHAYRCADILFNFCERRCTPAAVAASTK

SEQ ID NO: 51 - DNA, 731 bp, Zea mays
  misc_feature (0)...(0): VP-68 ZM-IT6_entire NT seq
cagaaacaaacaaaaaaacacccgggaaatccaagcagctgttgcaaccggttccttcgaggagaacgtgagaaagaagcatgaggccccaggtgctgctcgtcgcactggccgtcttcgccgtcctcgtagctctgccactgggcaaagcgcacgaggaggaggaggaggaagggcttgaactcgaagccagcaggaggcggtggccgtgctgcgaccagtgcggcatttgcaccaggtcgcagccgccgatatgcgagtgcagggacacgtcgacgaccggctgccacccggcttgcaaggcgtgcgccctgtccatctccgacggcctcttcgtgtgcaaggacaagatcgtcaacttctgcaagcgccgctgcacccgtcgtactgatgatgatgatgcgtgacttataatctaagtgggcaaataaccttgccaataaataatttccgttcggagctccatttgcatctttctcaagatgcagacgacagggtttaaataatcaaggcgatgcttgcttgctcgtcgcctttttactcgtacagtttctgaaataaagagcatgggtatgttatgctcggagatatatgtagtcatgagacgtaaaagaatgcaatatacgctttggttatgtgcatctttagatgccatagaccgaggcatgattatttcgcggtgatagctaataacgcatggctactttagcgtcaaaaaaaaaaaaaaaaaa

SEQ ID NO: 52 - DNA, 255 bp, Zea mays
  misc_feature (0)...(0): VP-68 ZM-IT6_coding region for mature protein
cacgaggaggaggaggaggaagggcttgaactcgaagccagcaggaggcggtggccgtgctgcgaccagtgcggcatttgcaccaggtcgcagccgccgatatgcgagtgcagggacacgtcgacgaccggctgccacccggcttgcaaggcgtgcgccctgtccatctccgacggcctcttcgtgtgcaaggacaagatcgtcaacttctgcaagcgccgctgcacccgtcgtactgatgatgatgatgcgtga

SEQ ID NO: 53 - protein, 108 aa, Zea mays
  PEPTIDE (0)...(0): VP-68 ZM-IT6_complete predicted protein
MRPQVLLVALAVFAVLVALPLGKAHEEEEEEGLELEASRRRWPCCDQCGICTRSQPPICECRDTSTTGCHPACKACALSISDGLFVCKDKIVNFCKRRCTRRTDDDDA

SEQ ID NO: 54 - protein, 84 aa, Zea mays
  PEPTIDE (0)...(0): VP-68 ZM-IT6_predicted mature protein
HEEEEEEGLELEASRRRWPCCDQCGICTRSQPPICECRDTSTTGCHPACKACALSISDGLFVCKDKIVNFCKRRCTRRTDDDDA

SEQ ID NO: 55 - DNA, 482 bp, Amaranthus retroflexus
  misc_feature (0)...(0): VP-74 AR-PDF1_entire NT seq
gcacgaggatcaagatcctcaaattaatcaatgaagacatttggagctttcgttcttatttttcttcttgcatccttcgccataacagggccaagaatgacggaagcaaggatgtgcaaagctccgagcaaactgtttaggggaatgtgtggtattagggattccaactgtgatagtgtttgcagggcggaaggaatggctgctggagattgccatggccttcgtagacgatgcatttgcagcaggccttgtccttaaattaccttatgtaatctcctaaaaataatgataacaaatgtttctatcttcaccattagctttaattattatcacctggctagtagctacatgcatataatgtaatcttatatagcgtcttgctatcactctatctctatgtttaaataatttcgtcttttatgtattaattgtttcttttcacatctataaattaacgataagatatctgtattcgtacactt

SEQ ID NO: 56 - DNA, 228 bp, Amaranthus retroflexus
  misc_feature (0)...(0): VP-74 AR-PDF1_coding region for complete protein
atgaagacatttggagctttcgttcttatttttcttcttgcatccttcgccataacagggccaagaatgacggaagcaaggatgtgcaaagctccgagcaaactgtttaggggaatgtgtggtattagggattccaactgtgatagtgtttgcagggcggaaggaatggctgctggagattgccatggccttcgtagacgatgcatttgcagcaggccttgtccttaa

SEQ ID NO: 57 - DNA, 150 bp, Amaranthus retroflexus
  misc_feature (0)...(0): VP-74 AR-PDF1_coding region for mature protein
aggatgtgcaaagctccgagcaaactgtttaggggaatgtgtggtattagggattccaactgtgatagtgtttgcagggcggaaggaatggctgctggagattgccatggccttcgtagacgatgcatttgcagcaggccttgtccttaa

SEQ ID NO: 58 - protein, 75 aa, Amaranthus retroflexus
  PEPTIDE (0)...(0): VP-74 AR-PDF1_complete predicted protein
MKTFGAFVLIFLLASFAITGPRMTEARMCKAPSKLFRGMCGIRDSNCDSVCRAEGMAAGDCHGLRRRCICSRPCP

SEQ ID NO: 59 - protein, 49 aa, Amaranthus retroflexus
  PEPTIDE (0)...(0): VP-74 AR-PDF1_predicted mature protein
RMCKAPSKLFRGMCGIRDSNCDSVCRAEGMAAGDCHGLRRRCICSRPCP

SEQ ID NO: 60 - DNA, 802 bp, Zea mays
  misc_feature (0)...(0): VP-94 ZM-TAI1_entire NT seq
gttcgagaagactccatcaagcagctcatacgtatatcacttgccatgccagcctagcttcttccgtgtctttctttctcaataaatacatgccactcacatggtgctcctccatcttccaagtcaccaacgcaaattgcaccaagaaatccatcgagaggccgtcgacaggggaattaatggcgtcgtcgtctagcagcagccaccgccgcctcatcctcgcagccgccgtcctgctctccgtgctcgcggctgccagcgccagcgccgggacctcctgcgtgccggggtgggccatcccgcacaacccgctcccgagctgccgctggtacgtgaccagccggacctgcggcatcgggccgcgcctcccgtggccggagctgaagaggagatgctgccgggagctggcggacatcccggcgtactgccggtgcacggcgctgagcatcctcatggacggcgcgatcccgccgggcccggacgcgcagctggagggccgcctagaggacctgccgggctgcccgcgggaggtgcagaggggattcgccgccaccctcgtcacggaggccgagtgcaacctggccaccatcagcggcgtcgccgaatgcccctggattctcggcggcggaacgatgccctccaagtaactgcgaagagcatagtgcatgaggaatgagcttgtagctagctcatatgtctgaataataagcacagcaagaagatgaatgcatttctcggatcgttcatccggaacaataattaaaggggatccggatttgttcttgtgaaaaaaaaaaaaaaa

SEQ ID NO: 61 - DNA, 468 bp, Zea mays
  misc_feature (0)...(0): VP-94 ZM-TAI1_coding region for complete protein
atggcgtcgtcgtctagcagcagccaccgccgcctcatcctcgcagccgccgtcctgctctccgtgctcgcggctgccagcgccagcgccgggacctcctgcgtgccggggtgggccatcccgcacaacccgctcccgagctgccgctggtacgtgaccagccggacctgcggcatcgggccgcgcctcccgtggccggagctgaagaggagatgctgccgggagctggcggacatcccggcgtactgccggtgcacggcgctgagcatcctcatggacggcgcgatcccgccgggcccggacgcgcagctggagggccgcctagaggacctgccgggctgcccgcgggaggtgcagaggggattcgccgccaccctcgtcacggaggccgagtgcaacctggccaccatcagcggcgtcgccgaatgcccctggattctcggcggcggaacgatgccctccaagtaa

SEQ ID NO: 62 - DNA, 384 bp, Zea mays
  misc_feature (0)...(0): VP-94 ZM-TAI1_coding region for mature protein
agcgccgggacctcctgcgtgccggggtgggccatcccgcacaacccgctcccgagctgccgctggtacgtgaccagccggacctgcggcatcgggccgcgcctcccgtggccggagctgaagaggagatgctgccgggagctggcggacatcccggcgtactgccggtgcacggcgctgagcatcctcatggacggcgcgatcccgccgggcccggacgcgcagctggagggccgcctagaggacctgccgggctgcccgcgggaggtgcagaggggattcgccgccaccctcgtcacggaggccgagtgcaacctggccaccatcagcggcgtcgccgaatgcccctggattctcggcggcggaacgatgccctccaagtaa

SEQ ID NO: 63 - protein, 155 aa, Zea mays
  PEPTIDE (0)...(0): VP-94 ZM-TAI1_complete predicted protein
MASSSSSSHRRLILAAAVLLSVLAAASASAGTSCVPGWAIPHNPLPSCRWYVTSRTCGIGPRLPWPELKRRCCRELADIPAYCRCTALSILMDGAIPPGPDAQLEGRLEDLPGCPREVQRGFAATLVTEAECNLATISGVAECPWILGGGTMPSK

SEQ ID NO: 64 - protein, 127 aa, Zea mays
  PEPTIDE (0)...(0): VP-94 ZM-TAI1_predicted mature protein
SAGTSCVPGWAIPHNPLPSCRWYVTSRTCGIGPRLPWPELKRRCCRELADIPAYCRCTALSILMDGAIPPGPDAQLEGRLEDLPGCPREVQRGFAATLVTEAECNLATISGVAECPWILGGGTMPSK

SEQ ID NO: 65 - DNA, 646 bp, Zea mays
  misc_feature (0)...(0): VP95 ZM-TAI3_entire NT seq
aagaagtcgatggggcacggaggagcagcaatggcgtcgtccagccacaccctcctcagcgctgcggttctgctgtccgtcctagccgccgccgctgccaggaccaactggtgcgagccagggctggtcatcccgctcaacccgctccagagttgccgcgcgtacctggttcgccggacctgcggcctcggccgcggccccttcgtgcccctgccggtgctcaagcagaggtgctgcacggagctggaggagatcgtgccctactgccggtgcggcgcgctgagggtcatgatggacggtatgccgggcggcggcgtggacaggccgccctgctcttggggcgggcagctggagctcgcgccgaccctcgtgtcggaggcggagtgcaacctggtcaccatccacggccgcccgttctgctacgcgctcggagctgaaggaacaacgacggattaggtttccgtaaatgtatatgatgccgccggtgtgtactgaataataagggggaacaagctgtgctcttgttattttctttaatttgaaagtgacccataaaaaagatgttcgtgttgggttgtgagttgtgacacacaggcggaagctggttggccggttgcctttaaaaaaaaaaaaaaaaaaaaaaaaa

SEQ ID NO: 66 - DNA, 426 bp, Zea mays
  misc_feature (0)...(0): ZM-TAI3_coding region for compete protein
atggcgtcgtccagccacaccctcctcagcgctgcggttctgctgtccgtcctagccgccgccgctgccaggaccaactggtgcgagccagggctggtcatcccgctcaacccgctccagagttgccgcgcgtacctggttcgccggacctgcggcctcggccgcggccccttcgtgcccctgccggtgctcaagcagaggtgctgcacggagctggaggagatcgtgccctactgccggtgcggcgcgctgagggtcatgatggacggtatgccgggcggcggcgtggacaggccgccctgctcttggggcgggcagctggagctcgcgccgaccctcgtgtcggaggcggagtgcaacctggtcaccatccacggccgcccgttctgctacgcgctcggagctgaaggaacaacgacggattag

SEQ ID NO: 67 - DNA, 357 bp, Zea mays
  misc_feature (0)...(0): VP-95 ZM-TAI3_coding region for complete protein
aggaccaactggtgcgagccagggctggtcatcccgctcaacccgctccagagttgccgcgcgtacctggttcgccggacctgcggcctcggccgcggccccttcgtgcccctgccggtgctcaagcagaggtgctgcacggagctggaggagatcgtgccctactgccggtgcggcgcgctgagggtcatgatggacggtatgccgggcggcggcgtggacaggccgccctgctcttggggcgggcagctggagctcgcgccgaccctcgtgtcggaggcggagtgcaacctggtcaccatccacggccgcccgttctgctacgcgctcggagctgaaggaacaacgacggattag

SEQ ID NO: 68 - protein, 141 aa, Zea mays
  PEPTIDE (0)...(0): VP-95 ZM-TAI3_complete predicted protein
MASSSHTLLSAAVLLSVLAAAAARTNWCEPGLVIPLNPLQSCRAYLVRRTCGLGRGPFVPLPVLKQRCCTELEEIVPYCRCGALRVMMDGMPGGGVDRPPCSWGGQLELAPTLVSEAECNLVTIHGRPFCYALGAEGTTTD

SEQ ID NO: 69 - protein, 118 aa, Zea mays
  PEPTIDE (0)...(0): VP-95 ZM-TAI3_complete mature protein
RTNWCEPGLVIPLNPLQSCRAYLVRRTCGLGRGPFVPLPVLKQRCCTELEEIVPYCRCGALRVMMDGMPGGGVDRPPCSWGGQLELAPTLVSEAECNLVTIHGRPFCYALGAEGTTTD

SEQ ID NO: 70 - DNA, 581 bp, Zea mays
  misc_feature (0)...(0): VP-122 ZM-ISC4_entire NT seq
gatccattaacactactcagcgcagctgtacgacacgagagatcaaatatatatggggcgcggcggtctgtcggcaaggagcgccaccgccgtcgtcgtcgacggtggggacaataaggagctgaagtcgtcgtggccggagacggtcgggaagcacatggtggaggcgatcgccatcatcaagtcggagaggcaggacgtcatcctggagctccacaccgccgacgacccgcagccgccggactacaacgaccagcgcgtctgcctcttcctcgacgacagcctgatcgtcgtcaggacgcccgtcgtcggctagctgctggccgggccgacggtcgtttccttcaacctagctagctacgggtacacagattaaaggacgatgtgtatgtaggattttgttgttgtactttacgtgtgtgtgacgatgtgatgagatcgtgtgatcgatcataataagtatttaccgtacgtacgtgatctgcatgcatgcatggcatgcgagtgtaccagtagagagtgtgcaagaataatgctgtacccgaccaaaaaaaaaaaaaaaaaaaaaaaaaaaaaaaaaa

SEQ ID NO: 71 - DNA, 264 bp, Zea mays
  misc_feature (0)...(0): VP-122 ZM-ISC4_coding region for complete protein
atggggcgcggcggtctgtcggcaaggagcgccaccgccgtcgtcgtcgacggtggggacaataaggagctgaagtcgtcgtggccggagacggtcgggaagcacatggtggaggcgatcgccatcatcaagtcggagaggcaggacgtcatcctggagctccacaccgccgacgacccgcagccgccggactacaacgaccagcgcgtctgcctcttcctcgacgacagcctgatcgtcgtcaggacgcccgtcgtcggctag

SEQ ID NO: 72 - DNA, 264 bp, Zea mays; identical to SEQ ID NO: 71
  misc_feature (0)...(0): VP-122 ZM-ISC4_coding region for mature protein

SEQ ID NO: 73 - protein, 87 aa, Zea mays
  PEPTIDE (0)...(0): VP-122 ZM-ISC4_complete predicted protein
MGRGGLSARSATAVVVDGGDNKELKSSWPETVGKHMVEAIAIIKSERQDVILELHTADDPQPPDYNDQRVCLFLDDSLIVVRTPVVG

SEQ ID NO: 74 - protein, 87 aa, Zea mays; identical to SEQ ID NO: 73
  PEPTIDE (0)...(0): VP-122 ZM-ISC4_predicted mature protein

SEQ ID NO: 75 - DNA, 995 bp, Zea mays
  misc_feature (0)...(0): VP-125 ZM-IT4_entire NT seq
caggagcaagaacaacaggaagagtcgaagagaggagatcagggacaagaacagccagaaagaggagaggaggctcatcaatcgcaactaattctcgcagtcgcagagacaagcaatcatcagacatgagaacccaggcgctgttcttcctcgcactcgcacttatcggcgccgtcctcgcgcagtccagcaacaggcaccaccaccactaccaccacgcccatgtccaaggcaaagggcagggagccggaggaggaggaggggagctggcgagccgggcgaaggcggcggcgcgcgcgtggccgtgctgtgacagctgcggcgggtgcaccagatcggagccgccgcggtgccagtgcctggacgcggcgccccgcgggtgccacccggcctgcagggactgcgtcaagtccagcctcagcgccgacccgccggtgtaccagtgcatggaccgcgtccccaacttctgccagcgccgctgcaccgccgccgccgccgccgcggcgcactgaccgaccgcgcggggcagccgagccagccgacgactggtttctgcttgcagcttggtattattgacccagcaacctttttgcccacccacccacccgctcgcccgcccgccggcgaatcatggcgtctggagcctttgaagccagccgtggattaggatttttgtttgtttgtgtttgtgtagactgtaactgtaaagagagatgaggaatctgtaatctggtgaacgagcaaccaatccgagcttacgttgtccgtctgtggtgtggtgtccttcttcttcttcttgtgttctgttttctttcattcagttacacactttggtaggtagttggcactgctttgctctgtatggctgactcttgttgtctgtgtgagagagatgagcatgatggatcactgagatttaaaaaaaaaaaaaaaaaaaaaaaaaaaaaaaaaaaaaaaaaaaaaaaaaaaaaaaaaaaaaaaaaaaaaaaaaaaaa

SEQ ID NO: 76 - DNA, 387 bp, Zea mays
  misc_feature (0)...(0): VP-125 ZM-IT4_coding region for complete protein
atgagaacccaggcgctgttcttcctcgcactcgcacttatcggcgccgtcctcgcgcagtccagcaacaggcaccaccaccactaccaccacgcccatgtccaaggcaaagggcagggagccggaggaggaggaggggagctggcgagccgggcgaaggcggcggcgcgcgcgtggccgtgctgtgacagctgcggcgggtgcaccagatcggagccgccgcggtgccagtgcctggacgcggcgccccgcgggtgccacccggcctgcagggactgcgtcaagtccagcctcagcgccgacccgccggtgtaccagtgcatggaccgcgtccccaacttctgccagcgccgctgcaccgccgccgccgccgccgcggcgcactga

SEQ ID NO: 77 - DNA, 330 bp, Zea mays
  misc_feature (0)...(0): VP-125 ZM-IT4_coding region for mature protein
cagtccagcaacaggcaccaccaccactaccaccacgcccatgtccaaggcaaagggcagggagccggaggaggaggaggggagctggcgagccgggcgaaggcggcggcgcgcgcgtggccgtgctgtgacagctgcggcgggtgcaccagatcggagccgccgcggtgccagtgcctggacgcggcgccccgcgggtgccacccggcctgcagggactgcgtcaagtccagcctcagcgccgacccgccggtgtaccagtgcatggaccgcgtccccaacttctgccagcgccgctgcaccgccgccgccgccgccgcggcgcactga

SEQ ID NO: 78 - protein, 128 aa, Zea mays
  PEPTIDE (0)...(0): VP-125 ZM-IT4_complete predicted protein
MRTQALFFLALALIGAVLAQSSNRHHHHYHHAHVQGKGQGAGGGGGELASRAKAAARAWPCCDSCGGCTRSEPPRCQCLDAAPRGCHPACRDCVKSSLSADPPVYQCMDRVPNFCQRRCTAAAAAAAH

SEQ ID NO: 79 - protein, 109 aa, Zea mays
  PEPTIDE (0)...(0): VP-125 ZM-IT4_predicted mature protein
QSSNRHHHHYHHAHVQGKGQGAGGGGGELASRAKAAARAWPCCDSCGGCTRSEPPRCQCLDAAPRGCHPACRDCVKSSLSADPPVYQCMDRVPNFCQRRCTAAAAAAAH

SEQ ID NO: 80 - DNA, 923 bp, Zea mays
  misc_feature (0)...(0): VP-126 ZM-IT5_entire NT seq
cttcaggcttggagaaagaagaaaggcatagcagcgggcagcggcatgggcaccagccccgtcatccctgcgacgatgctcttcgtcgctcttcttgtcgctgtctgcttcggcgccaccgccgccaggtccagccacgcccgccacaccgacgccgacgacgcgattcgtctccccagcagctttggcgctggtggtcgaccgtggaattgctgcgactccgctatcaaggacgcctccctgccggcgtggcgctgcaacgacgtggtggacaagtgctccgccgactgccaggactgcgaggcgtcgccggccggcgacggcttcgtctgcggtgactttatcttaagcctgaccgagcccccggtctgcacaccgaggccgtgggactgctgcgacagcgtggcctgcacaagggactacatccccaactgcatgtgcctggacaaggtggagtcgtgctcgagcaactgcgaacggtgcgagtttctgtatcattcggaccctcctcgcttccaatgccgagacatcttccacggctatcctggtcccaagtgcaggacatggatcagtacgagcaactagctcagctcagctacttgctaaaagtagcgtagcgcagcgcgcgcggctacttgctagtatacgtacgtccacaaataaaaataaaagtggctagcgtacaaagctaacatctggttgttcgtcccgacatggacgattagcctagctcgatcgctagctgctgccactctcgttcgatttaatttcatccccatgtgtggcggtgtagggagtgtggatatgatcatccaataaagtagtctactgaaaatgaatagtcgttgatcatattaatatacatccatctcattttgttcaaaaaaaaaaaaaaaaaaaaaaaaaaaaaaaaaaaaaaaaaa

SEQ ID NO: 81 - DNA, 540 bp, Zea mays
  misc_feature (0)...(0): VP-126 ZM-IT5_coding region for complete protein
atgggcaccagccccgtcatccctgcgacgatgctcttcgtcgctcttcttgtcgctgtctgcttcggcgccaccgccgccaggtccagccacgcccgccacaccgacgccgacgacgcgattcgtctccccagcagctttggcgctggtggtcgaccgtggaattgctgcgactccgctatcaaggacgcctccctgccggcgtggcgctgcaacgacgtggtggacaagtgctccgccgactgccaggactgcgaggcgtcgccggccggcgacggcttcgtctgcggtgactttatcttaagcctgaccgagcccccggtctgcacaccgaggccgtgggactgctgcgacagcgtggcctgcacaagggactacatccccaactgcatgtgcctggacaaggtggagtcgtgctcgagcaactgcgaacggtgcgagtttctgtatcattcggaccctcctcgcttccaatgccgagacatcttccacggctatcctggtcccaagtgcaggacatggatcagtacgagcaactag

SEQ ID NO: 82 - DNA, 462 bp, Zea mays
  misc_feature (0)...(0): VP-126 ZM-IT5_coding region for mature protein
gccaggtccagccacgcccgccacaccgacgccgacgacgcgattcgtctccccagcagctttggcgctggtggtcgaccgtggaattgctgcgactccgctatcaaggacgcctccctgccggcgtggcgctgcaacgacgtggtggacaagtgctccgccgactgccaggactgcgaggcgtcgccggccggcgacggcttcgtctgcggtgactttatcttaagcctgaccgagcccccggtctgcacaccgaggccgtgggactgctgcgacagcgtggcctgcacaagggactacatccccaactgcatgtgcctggacaaggtggagtcgtgctcgagcaactgcgaacggtgcgagtttctgtatcattcggaccctcctcgcttccaatgccgagacatcttccacggctatcctggtcccaagtgcaggacatggatcagtacgagcaactag

SEQ ID NO: 83 - protein, 179 aa, Zea mays
  PEPTIDE (0)...(0): VP-126 ZM-IT5_complete predicted protein
MGTSPVIPATMLFVALLVAVCFGATAARSSHARHTDADDAIRLPSSFGAGGRPWNCCDSAIKDASLPAWRCNDVVDKCSADCQDCEASPAGDGFVCGDFILSLTEPPVCTPRPWDCCDSVACTRDYIPNCMCLDKVESCSSNCERCEFLYHSDPPRFQCRDIFHGYPGPKCRTWISTSN

SEQ ID NO: 84 - protein, 153 aa, Zea mays
  PEPTIDE (0)...(0): VP-126 ZM-IT5_predicted mature protein
ARSSHARHTDADDAIRLPSSFGAGGRPWNCCDSAIKDASLPAWRCNDVVDKCSADCQDCEASPAGDGFVCGDFILSLTEPPVCTPRPWDCCDSVACTRDYIPNCMCLDKVESCSSNCERCEFLYHSDPPRFQCRDIFHGYPGPKCRTWISTSN

SEQ ID NO: 85 - DNA, 478 bp, Nicotiana benthamiana
  misc_feature (0)...(0): VP-133 NB-PDF2_entire NT seq
aacaaattaaagaattttaagaaaaacaagatggctggctttcccaaagtgcttgcaactgttttccttacgctgatgctggtttttgctactgagatgggaccaatggtgactgaggcgaggacctgcgagtcacagagccaccgattcaagggtttgtgtttcagtaggagcaactgtgcgtctgtttgccatactgagggctttaacggtggccactgccgtggattccgtcgccgttgcttctgcaccagacactgttaattattattattatgtgtactgctgtgtaatatgaacgtctcttctcgtttcttctggtgtttgtcatgaaataagaatgaccatctgaactcagaaacagatcagaatggttaattcccttccgtttcctangagttaaatggttgctggcaacttttaattgcgaactctttctgtaactattgggtattacgatatattaaaaaaaaaacca

SEQ ID NO: 86 - DNA, 234 bp, Nicotiana benthamiana
  misc_feature (0)...(0): VP-133 NB-PDF2_coding region for complete protein
atggctggctttcccaaagtgcttgcaactgttttccttacgctgatgctggtttttgctactgagatgggaccaatggtgactgaggcgaggacctgcgagtcacagagccaccgattcaagggtttgtgtttcagtaggagcaactgtgcgtctgtttgccatactgagggctttaacggtggccactgccgtggattccgtcgccgttgcttctgcaccagacactgttaa

SEQ ID NO: 87 - DNA, 144 bp, Nicotiana benthamiana
  misc_feature (0)...(0): VP-133 NB-PDF2_coding region for mature protein
aggacctgcgagtcacagagccaccgattcaagggtttgtgtttcagtaggagcaactgtgcgtctgtttgccatactgagggctttaacggtggccactgccgtggattccgtcgccgttgcttctgcaccagacactgttaa

SEQ ID NO: 88 - protein, 77 aa, Nicotiana benthamiana
  PEPTIDE (0)...(0): VP-133 NB-PDF2_complete predicted protein
MAGFPKVLATVFLTLMLVFATEMGPMVTEARTCESQSHRFKGLCFSRSNCASVCHTEGFNGGHCRGFRRRCFCTRHC

SEQ ID NO: 89 - protein, 47 aa, Nicotiana benthamiana
  PEPTIDE (0)...(0): VP-133 NB-PDF2_predicted mature protein
RTCESQSHRFKGLCFSRSNCASVCHTEGFNGGHCRGFRRRCFCTRHC

SEQ ID NO: 90 - DNA, 629 bp, Triticum aestivum
  misc_feature (0)...(0): VP-139 TA-PDF26_entire NT seq
gcacgaggctagacaggacagcctatattgacgtagacgcagcaggagcagcagcagcgaactctgtcagttctctagcatctccggtgaagcaagcaagcagagagatggcgtcccctcgtcgcatggccgccgcgcccgccgtcctcctcctcgtcctgctcctcctcgtcgccacggagatggggacgatgaagacggcggaggcccggacgtgcctgtcgcagagccacaagttcaagggcacctgcctcagcaacagcaactgcgccggcgtgtgccgcaccgagaacttccccgacggcgagtgcaactcccaccgcctcgagcgcaagtgcttctgcaagcgcacctgctaagcaagcccagtccgcgctactggctctggctagctagactgctagatcagcagccatgccgtcagttagatctgttcgtccctacttttgtttccgtttgctttacgttgctcttggggatgactgaaaataaagtagctacctacatcctctgcattggctgttccactgcatgttgtctaagtgtttctggctttagtttgtgctgttgatgtaataacgatgccactaacaatttggcttctatgtgttgtgttgaaacttggaatc

SEQ ID NO: 91 - DNA, 252 bp, Triticum aestivum
  misc_feature (0)...(0): VP-139 TA-PDF26_coding region for complete protein
atggcgtcccctcgtcgcatggccgccgcgcccgccgtcctcctcctcgtcctgctcctcctcgtcgccacggagatggggacgatgaagacggcggaggcccggacgtgcctgtcgcagagccacaagttcaagggcacctgcctcagcaacagcaactgcgccggcgtgtgccgcaccgagaacttccccgacggcgagtgcaactcccaccgcctcgagcgcaagtgcttctgcaagcgcacctgctaa

SEQ ID NO: 92 - DNA, 150 bp, Triticum aestivum
  misc_feature (0)...(0): VP-139 TA-PDF26_coding region for mature protein
cggacgtgcctgtcgcagagccacaagttcaagggcacctgcctcagcaacagcaactgcgccggcgtgtgccgcaccgagaacttccccgacggcgagtgcaactcccaccgcctcgagcgcaagtgcttctgcaagcgcacctgctaa

SEQ ID NO: 93 - protein, 83 aa, Triticum aestivum
  PEPTIDE (0)...(0): VP-139 TA-PDF26_complete predicted protein
MASPRRMAAAPAVLLLVLLLLVATEMGTMKTAEARTCLSQSHKFKGTCLSNSNCAGVCRTENFPDGECNSHRLERKCFCKRTC

SEQ ID NO: 94 - protein, 49 aa, Triticum aestivum
  PEPTIDE (0)...(0): VP-139 TA-PDF26_predicted mature protein
RTCLSQSHKFKGTCLSNSNCAGVCRTENFPDGECNSHRLERKCFCKRTC

SEQ ID NO: 95 - DNA, 537 bp, Tulipa gesneriana
  misc_feature (0)...(0): VP-140 TG-PDF1_entire NT seq
ggtcttccagtactccttacccttcactatggcgaagcttcccacagtcctcctgctcctcttcctcgtcgtggcctttgaaatgggaacgacgacggtggaggcgaggacgtgcctgtcacagagccacaagttcgaaggtacctgcctgagggagtccaactgcgcgaccgtctgccagacggaggggttccatggaggagggacttgccagggcttccgccgccgctgcttctgcgtaagaaactgttgatgatgcttcatgcttcgaatctattcggagcagtatggcaagagcccatggtgctctatttcatgggtttatcaagtgttttgagatgaataagatggaccttagtgtgcttgcttcacctaagttcaagtgtttcctgcctaaatttgtataatttgtttgagtttccgtcnactgtcctctgtantttggagccggttgtacggtaacatttaatatcaaaaanttccaantttaaaaaaaaaaaaaaaaaacccnanggggggggccggtancaaattccc

SEQ ID NO: 96 - DNA, 225 bp, Tulipa gesneriana
  misc_feature (0)...(0): VP-140 TG-PDF1_entire NT seq
atggcgaagcttcccacagtcctcctgctcctcttcctcgtcgtggcctttgaaatgggaacgacgacggtggaggcgaggacgtgcctgtcacagagccacaagttcgaaggtacctgcctgagggagtccaactgcgcgaccgtctgccagacggaggggttccatggaggagggacttgccagggcttccgccgccgctgcttctgcgtaagaaactgttga

SEQ ID NO: 97 - DNA, 147 bp, Tulipa gesneriana
  misc_feature (0)...(0): VP-140 TG-PDF1_coding region for mature protein
aggacgtgcctgtcacagagccacaagttcgaaggtacctgcctgagggagtccaactgcgcgaccgtctgccagacggaggggttccatggaggagggacttgccagggcttccgccgccgctgcttctgcgtaagaaactgttga

SEQ ID NO: 98 - protein, 74 aa, Tulipa gesneriana
  PEPTIDE (0)...(0): VP-140 TG-PDF1_complete predicted protein
MAKLPTVLLLLFLVVAFEMGTTTVEARTCLSQSHKFEGTCLRESNCATVCQTEGFHGGGTCQGFRRRCFCVRNC

SEQ ID NO: 99 - protein, 48 aa, Tulipa gesneriana
  PEPTIDE (0)...(0): VP-140 TG-PDF1_predicted mature protein
RTCLSQSHKFEGTCLRESNCATVCQTEGFHGGGTCQGFRRRCFCVRNC

SEQ ID NO: 100 - DNA, 441 bp, Tulipa gesneriana
  misc_feature (0)...(0): VP-142 TG-PDF3_entire NT seq
gacctcaccaagctagtcttccancacgcctctgccttcagatatgtcgaaaattccgaccctcctactgctgctcgtcctcgttgtcgcctcagaaatggggacaacgacggtggaggcggattgctacagaccgagcgggagttaccacggcccctgctttgattcggacggctgcgatagtacctgcaagattcaggacgggaaaccaggagggacttgcagcggcttccgctgcttctgcaactgttgatgatgcttcaagcttcnaatctattcggagaaatgtggtanaaccttgtggtgctctatttcaagggtttaataaagtgtcttcatattaaaaaaatgggccttaagtgtgcttcacctaaggttcaatgcttttcctgcttaaatgtgttgtagcttgtgtgagcttcnacgtccgtgtcaagttgg

SEQ ID NO: 101 - DNA, 210 bp, Tulipa gesneriana
  misc_feature (0)...(0): VP-142 TG-PDF3_coding region for complete protein
atgtcgaaaattccgaccctcctactgctgctcgtcctcgttgtcgcctcagaaatggggacaacgacggtggaggcggattgctacagaccgagcgggagttaccacggcccctgctttgattcggacggctgcgatagtacctgcaagattcaggacgggaaaccaggagggacttgcagcggcttccgctgcttctgcaactgttga

SEQ ID NO: 102 - DNA, 132 bp, Tulipa gesneriana
  misc_feature (0)...(0): VP-142 TG-PDF3_coding region for mature protein
gattgctacagaccgagcgggagttaccacggcccctgctttgattcggacggctgcgatagtacctgcaagattcaggacgggaaaccaggagggacttgcagcggcttccgctgcttctgcaactgttga

SEQ ID NO: 103 - protein, 69 aa, Tulipa gesneriana
  PEPTIDE (0)...(0): VP-142 TG-PDF3_complete predicted protein
MSKIPTLLLLLVLVVASEMGTTTVEADCYRPSGSYHGPCFDSDGCDSTCKIQDGKPGGTCSGFRCFCNC

SEQ ID NO: 104 - protein, 43 aa, Tulipa gesneriana
  PEPTIDE (0)...(0): VP-142 TG-PDF3_predicted mature protein
DCYRPSGSYHGPCFDSDGCDSTCKIQDGKPGGTCSGFRCFCNC

SEQ ID NO: 105 - DNA, 469 bp, Beta vulgaris
  misc_feature (0)...(0): VP-152 BV-PDF2_entire NT seq
gcacgaggagaaaacataaaatggaacgctcttcacgtctgttttcagctgttcttcttgtgcttctgcttgtgatttctacagaggttggaaccaaggtggtagaagcaagaatatgtgagtcaccaagttacaggttcaggggaatttgtgtgagcaggaacaactgtgctaatatctgcaaaactgaaggttttcccggtggccgttgccgcggtttccgccgtcgttgcttctgttacaaacactgcgcctaattgtagtactccatgcatttctgatcaagtgctagtagtacactatgcaattcatatatgtgttatgttacataaatgaagtgccttctttaattacctactatggtttttgtaatctttaagaataagttcagttgtaatcgttgtcatttgcatatcatattagttatggttaattttaatgtatgatctttaatttgagttgtcaannn

SEQ ID NO: 106 - DNA, 237 bp, Beta vulgaris
  misc_feature (0)...(0): VP-152 BV-PDF2_coding region for complete protein
atggaacgctcttcacgtctgttttcagctgttcttcttgtgcttctgcttgtgatttctacagaggttggaaccaaggtggtagaagcaagaatatgtgagtcaccaagttacaggttcaggggaatttgtgtgagcaggaacaactgtgctaatatctgcaaaactgaaggttttcccggtggccgttgccgcggtttccgccgtcgttgcttctgttacaaacactgcgcctaa

SEQ ID NO: 107 - DNA, 141 bp, Beta vulgaris
  misc_feature (0)...(0): VP-152 BV-PDF2_coding region for mature protein
agaatatgtgagtcaccaagttacaggttcaggggaatttgtgtgagcaggaacaactgtgctaatatctgcaaaactgaaggttttcccggtggccgttgccgcggtttccgccgtcgttgcttctgttacaaacactgc

SEQ ID NO: 108 - protein, 78 aa, Beta vulgaris
  PEPTIDE (0)...(0): VP-152 BV-PDF2_complete predicted protein
MERSSRLFSAVLLVLLLVISTEVGTKVVEARICESPSYRFRGICVSRNNCANICKTEGFPGGRCRGFRRRCFCYKHCA

SEQ ID NO: 109 - protein, 47 aa, Beta vulgaris
  PEPTIDE (0)...(0): VP-152 BV-PDF2_predicted mature protein
RICESPSYRFRGICVSRNNCANICKTEGFPGGRCRGFRRRCFCYKHC

SEQ ID NO: 110 - DNA, 470 bp, Hedera helix
  misc_feature (0)...(0): VP-153 HH-PDF1_entire NT seq
gcacgagggaagattaaatatggcaggaaaatttagcccaactagctttcttgcaatctctctcgtttttttccttctcgctaacacggaaacaattataggtgttgagggaaaattatgtgaaaaaccaagcttgacatggtccgggaaatgcggaaacacacagaactgtgataagcaatgccagacttgggaatctgcaaaacatggagcatgtcacaaacgaggcaattggaaatgcttctgttactttgactgttgatccaactccaaggaatatttaagaatcttaaaccatgcatgcataaaaatgcatgcgtatgagttaatttcctttgttattattagtactgcaatcttaataaataaaaggaaatgcttcttagctggcgcaaaaaaaaaaaaaaaaaaaaaaaaaaaaaaaaaaaaaaatttaaaaaaaaaaaaaaaaaaaaaaaaaaaaaaaaaaa

SEQ ID NO: 111 - DNA, 243 bp, Hedera helix
  misc_feature (0)...(0): VP-153 HH-PDF1_coding region for complete protein
atggcaggaaaatttagcccaactagctttcttgcaatctctctcgtttttttccttctcgctaacacggaaacaattataggtgttgagggaaaattatgtgaaaaaccaagcttgacatggtccgggaaatgcggaaacacacagaactgtgataagcaatgccagacttgggaatctgcaaaacatggagcatgtcacaaacgaggcaattggaaatgcttctgttactttgactgttga

SEQ ID NO: 112 - DNA, 147 bp, Hedera helix
  misc_feature (0)...(0): VP-153 HH-PDF1_coding region for mature protein
aaattatgtgaaaaaccaagcttgacatggtccgggaaatgcggaaacacacagaactgtgataagcaatgccagacttgggaatctgcaaaacatggagcatgtcacaaacgaggcaattggaaatgcttctgttactttgactgt

SEQ ID NO: 113 - protein, 80 aa, Hedera helix
  PEPTIDE (0)...(0): VP-153 HH-PDF1_complete predicted protein
MAGKFSPTSFLAISLVFFLLANTETIIGVEGKLCEKPSLTWSGKCGNTQNCDKQCQTWESAKHGACHKRGNWKCFCYFDC

SEQ ID NO: 114 - protein, 49 aa, Hedera helix
  PEPTIDE (0)...(0): VP-153 HH-PDF1_predicted mature protein
KLCEKPSLTWSGKCGNTQNCDKQCQTWESAKHGACHKRGNWKCFCYFDC

SEQ ID NO: 115 - DNA, 494 bp, Nicotiana benthamiana
  misc_feature (0)...(0): VP-168 NB-PDF3_entire NT seq
ctaagctgcctctctctgtcaaaaaatacttttgtctgtgaaaatggcaaaatccatgcgcttctttgccactgtgttacttctggcaatgcttgtcatggctactgagatgggaccaatgacagttgccgaggcaagacgttgcgagtcgaaaagccaacgttttaagggaccatgtgttagagtgaaaaattgtgccgccgtttgtgagaccgaaggattttccggtggtgactgccgtggactccgtcgccgttgtttttgtactaggccatgctaagaatgttactatatgttatatatgtaaaacctgaatttgagaaactattgaataagcattatgattgttcaacgattaacgtgctagtttgttactaattaaactatcgtgatctttgaccgttatgcaaatataangnacatttaagggggttgtgatttccaagggngaattcccgtgttccgcaacgttatggataaattctccttcaacc

SEQ ID NO: 116 - DNA, 237 bp, Nicotiana benthamiana
  misc_feature (0)...(0): NB-PDF3_coding region for complete protein
atggcaaaatccatgcgcttctttgccactgtgttacttctggcaatgcttgtcatggctactgagatgggaccaatgacagttgccgaggcaagacgttgcgagtcgaaaagccaacgttttaagggaccatgtgttagagtgaaaaattgtgccgccgtttgtgagaccgaaggattttccggtggtgactgccgtggactccgtcgccgttgtttttgtactaggccatgctaa

SEQ ID NO: 117 - DNA, 144 bp, Nicotiana benthamiana
  misc_feature (0)...(0): VP-168 NB-PDF3_coding region for mature protein
cggagatgcgagtccaagagccaaagattcaagggaccatgcgtgagagttaagaactgcgctgccgtttgtgaaaccgaaggattctcaggaggcgattgcagaggactgagacgcagatgcttctgtacaagaccttgctga

SEQ ID NO: 118 - protein, 86 aa, Nicotiana benthamiana
  PEPTIDE (0)...(0): VP-168 NB-PDF3_complete predicted protein
MPKYTAFIALILCLLLVAATEMQMAEGKYCWKKNHKWHGPCHYSYKCNHHCKHYFGAEYGVCKKYQWGHKHHHWAKYACYCYSPCH

SEQ ID NO: 119 - protein, 47 aa, Nicotiana benthamiana
  PEPTIDE (0)...(0): VP-168 NB-PDF3_predicted mature protein
RRCESKSQRFKGPCVRVKNCAAVCETEGFSGGDCRGLRRRCFCTRPC

SEQ ID NO: 120 - protein, 58 aa, Artificial Sequence
  Shuffled Peptide LB-9827-1A-2F3
LSKYGGECSLKHNTCTYRKGGKNQVVNCGTAANKKCKTDRHHCEYDEYHKRVDCQTPV

SEQ ID NO: 121 - protein, 58 aa, Artificial Sequence
  Shuffled Peptide LB-9827-2A-11E4
LSKYGGECSRKHNTCTYKKGGKNQIVNCPTAANKRCKTDRHHCEYDEYHRRVDCQTPV

SEQ ID NO: 122 - protein, 58 aa, Artificial Sequence
  Shuffled Peptide LB-9827-4F7
LSKYGGECSRKHNTCTYRKGGKNQVVKCPSAANKRCKTDRHHCEYDEYHRRVDCQTPV

SEQ ID NO: 123 - protein, 50 aa, Artificial Sequence
  Shuffled Peptide Pp-PDF1(7A4)
RVCEKPSKFFKGLCGSDRDCTNACRKEGLATGECQSKGFFNSVCVCKKPC

SEQ ID NO: 124 - protein, 50 aa, Artificial Sequence
  Shuffled Peptide Pp-PDF1(7C4)
RVCTKPSKFFKGMCVSDNDCTHACRKEGLATGFCQSKGFFNSVCVCTKPC